(19) United States
(12) Patent Application Publication
(10) Pub. No.: US 2024/0273361 A1
(43) Pub. Date: Aug. 15, 2024

(54) SYSTEMS AND METHODS FOR NEURAL NETWORKS AND DYNAMIC SPATIAL FILTERS TO REWEIGH CHANNELS

(71) Applicant: INTERAXON INC., Toronto (CA)

(72) Inventors: Hubert JACOB BANVILLE, Toronto (CA); Christopher Allen AIMONE, Scarborough (CA); Sean Ulrich Niethe WOOD, Toronto (CA)

(21) Appl. No.: 18/562,992

(22) PCT Filed: May 20, 2022
(86) PCT No.: PCT/CA2022/050820

§ 371 Date: Nov. 21, 2023

Related U.S. Application Data
(60) Provisional application No. 63/191,784, filed on May 21, 2021.

(51) Int. Cl. G06N 3/08; G06N 3/045

(52) U.S. Cl. CPC G06N 3/08; G06N 3/045

(57) ABSTRACT

Systems and methods disclosed herein are directed at the dynamic filtering of channels based on relevance of a channel to a learning task or channel corruption. In one aspect. a system is disclosed herein for dynamically reweighing a plurality of channels according to relevance given a learning task or channel corruption using a neural network. The system comprising a plurality of channels. each channel of the plurality of channels comprising data and a computing device. The computing device can be configured to receive a dataset from a plurality of channels. extract a representation of the dataset or the plurality of channels. predict a dynamic spatial filter from the representation of the dataset or the plurality of channels using a neural network. apply the dynamic spatial filter to dynamically reweigh each of the channels of plurality channels. and perform a learning task using the reweighed channels and a second neural network.

### FIELD

The methods, systems, and devices described herein generally relate to the field of signal processing, spatial filters, electrodes, channels, neural networks and, in particular, methods, systems, and devices described herein relate to reweighing channels according to their relevance to a learning task or their channel corruption using deep neural network architectures.

### INTRODUCTION

Machine learning processes can be used for electroencephalography (EEG) monitoring. Machine learning models used for different applications (e.g., EEG monitoring) need to be robust to noisy data and randomly missing channels, including when working with sparse montages (e.g., mobile EEG devices). However, deep neural networks trained end-to-end are not tested for robustness to corruption, especially to randomly missing channels. Accordingly, there may be additional challenges for sparse montages, signal quality conditions, and limited available computing power.

Lab and clinical applications can be translated to settings such as at-home or in remote locations. However, in these new settings, the number of sensors available can be limited and signal quality is harder to control. Moreover, the increasing availability of such devices (e.g., at-home or mobile) means the amount of data generated far exceeds the capacity of human experts (e.g., neurologists, sleep technicians, etc.) to analyze and manually annotate recordings, as traditionally done in research and clinical settings. Therefore, to expand the reach of these applications, there exists a need for automated, robust machine learning pipelines that can work with sparse montages or in challenging signal quality conditions.

### SUMMARY

Machine learning processes and encoded models can be used for electroencephalography (EEG) monitoring. Machine learning models used for different applications (e.g., EEG monitoring) need to be robust to noisy data and randomly missing channels, especially when working with sparse montages (e.g., mobile EEG devices). However, deep neural networks trained end-to-end are not tested for robustness to corruption, especially to randomly missing channels. There can be challenges when sparse montages are used and limited computing power is available.

Lab and clinical applications can be translated to settings such as at-home or in remote locations. However, in these new settings, the number of sensors available is often limited and signal quality is harder to control. Moreover, the increasing availability of such devices (e.g., at-home or mobile) means the amount of data generated far exceeds the capacity of human experts (e.g., neurologists, sleep technicians, etc.) to analyze and manually annotate recordings, as traditionally done in research and clinical settings. Therefore, to expand the reach of these applications, there exists a need for automated, robust machine learning pipelines that can work with sparse montages or in challenging signal quality conditions.

To alleviate this problem, embodiments described herein provide dynamic spatial filtering (DSF), which can involve a multi-head attention module that can be plugged before the first layer of a neural network (or subsequent layer of the neural network) to handle missing channels by learning to focus on good channels and ignore bad ones. DSF outputs can be interpretable, making it a useful tool also for monitoring effective channel importance and signal quality in real-time. This approach may enable analysis of channel data in challenging settings where channel corruption hampers brain signal readings.

In one aspect, embodiments described herein provide a method of using a neural network to dynamically reweigh a plurality of channels according to relevance given a learning task or channel corruption. The method involves receiving a dataset from a plurality of channels, each channel of the plurality of channels having data. The method involves extracting a representation of the dataset or the plurality of channels, predicting a dynamic spatial filter from the representation of the dataset or the plurality channels using a neural network, applying the dynamic spatial filter to dynamically reweigh each of the channels of the plurality channels, and performing a learning task using the reweighed channels and a second neural network.

In some embodiments, the dynamic spatial filter comprises unbounded weights to preserve the conceptual connection between channel recombination and spatial filtering. In some embodiments, neural network is trained for a predictive task.

In some embodiments, neural network is trained to perform a related learning task.

In some embodiments, the representation extracted from the dataset or plurality of channels comprises a first, second, third, or fourth order representation.

In some embodiments, the representation comprises a second order representation that comprises at least one of spatial covariance information, correlational information, and cosine similarity to capture dependencies between the plurality of channels.

In some embodiments, the method involves applying the dynamic spatial filter by applying the dynamic spatial filter to input of a first layer of the second neural network.

In some embodiments, the method involves applying the dynamic spatial filter by applying the dynamic spatial filter to output of layer of the second neural network.

In some embodiments, the representation is at least one of non-linear relational data between the channels such as fractal representations, mutual information, and Granger causality.

In some embodiments, the channels have a plurality of sensors and the method involves performing measurements for the dataset using the plurality of sensors of the channels.

In some embodiments, channels receive data from a plurality of subdivisions of a sensor.

In some embodiments, the dataset is made of output of a layer of the second neural network and the method involves performing a learning task using the reweighed channels and the second neural network involves providing the reweighed channels to at least one subsequent layer of the second neural network.

In some embodiments, the channels include bio-signal, EEG, EMG, fNIRS, audio-signal, seismographic, radar, telescopic, motion data, chemical sensor data, protein sensor data, and video-signal.

In some embodiments, channels can include an array of sensors and the method includes performing measurements for the dataset using the array of sensors of the channels. In some embodiments, the dynamic spatial filter includes a weight matrix.

In some embodiments, the dynamic spatial filter includes a bias vector.

In some embodiments, the method can also involve visualizing the dynamic spatial filter

in real-time at an interface.

In some embodiments, visualizing the dynamic spatial filter in real-time can include indicating a relative significance of at least one channel of the plurality of channels using one or more visual elements of the interface.

In some embodiments, the method involves visualizing the dynamic spatial filter in real-time by indicating signal quality feedback based in part on the learning task using one or more visual elements of the interface.

In some embodiments, the method further involves identifying an optimal location for hardware corresponding to at least one channel of the plurality of channels based in part on the dynamic spatial filter.

In some embodiments, the optimal location is determined in part by expected signals from an intended target of the at least one channel of the plurality of channels.

In some embodiments, the plurality of channels include a plurality of bio-signal sensors and the dataset aggregates bio-signal data from the bio-signal sensors. The learning task can involve predicting a brain state based in part on the bio-signal data, and the intended target can be a brain structure of a user and the method involves performing measurements for the bio-signal data using a plurality of bio-signal sensors of the channels.

In some embodiments, the predicting a dynamic spatial filter from the representation of the dataset or the plurality channels using a neural network involves soft-thresholding channels of the plurality channels.

In some embodiments, the method further involves identifying a source space using the dynamic spatial filter.

In some embodiments, the method further involves using results of the learning task to adjust at least one trainable parameter of at least one of the neural network and the second neural network.

In some embodiments, the applying the dynamic spatial filter involves adjusting the channels to a form acceptable by the second neural network in the performing a learning task.

In some embodiments, the method further involves selectively transmitting at least one channel of the plurality of channels based in part on the dynamic spatial filter.

In some embodiments, the applying the dynamic spatial filter involves selectively transmitting at least one dynamically reweighed channel.

In some embodiments, the learning task involves predicting a sleep stage.

In some embodiments, the learning task involves detecting pathologies.

In some embodiments, the plurality of channels includes a plurality of bio-signal sensors, and the dataset aggregates bio-signal data from the bio-signal sensors. The learning task can involve predicting a brain state based in part on the bio-signal data.

In one aspect, embodiments described herein provide a method of adjusting trainable parameters of neural networks to dynamically reweigh a plurality of channel according to relevance given a learning task or channel corruption. The method involves receiving a dataset from a plurality of channels, each channel of the plurality of channels having data. The method involves extracting a representation of the dataset or the plurality of channels, predicting a dynamic spatial filter from the representation of the dataset or the plurality of channels using a neural network, applying the dynamic spatial filter to dynamically reweigh each of the channels of the plurality of channels, performing a learning task using the reweighed channels and a second neural network, and using a learning objective to adjust at least one trainable parameter of at least one of the neural network and the second neural network.

In some embodiments, the dynamic spatial filter comprises unbounded weights to preserve the conceptual connection between channel recombination and spatial filtering.

In some embodiments, the learning objective involves minimizing a difference between predicted results and expected results.

In some embodiments, the representation extracted from the dataset or plurality of channels comprises a first, second, third, or fourth order representation.

In some embodiments, the representation comprises a second order representation that comprises at least one of spatial covariance information, correlational information, and cosine similarity to capture dependencies between the plurality of channels.

In some embodiments, the method involves applying the dynamic spatial filter by applying the dynamic spatial filter to input of a first layer of the second neural network.

In some embodiments, the method involves applying the dynamic spatial filter by applying the dynamic spatial filter to output of layer of the second neural network.

In some embodiments, the representation is at least one of non-linear relational data between the channels such as fractal representations, mutual information, and Granger causality.

In some embodiments, the channels have a plurality of sensors and the method involves performing measurements for the dataset using the plurality of sensors of the channels.

In some embodiments, channels receive data from a plurality of subdivisions of a sensor.

In some embodiments, the dataset is made of output of a layer of the second neural network and the method involves performing a learning task using the reweighed channels and the second neural network involves providing the reweighed channels to at least one subsequent layer of the second neural network.

In some embodiments, the channels include bio-signal, EEG, EMG, fNIRS, audio-signal, seismographic, radar, telescopic, motion data, chemical sensor data, protein sensor data, and video-signal.

In some embodiments, channels can include an array of sensors and the method includes performing measurements for the dataset using the array of sensors of the channels. In some embodiments, the dynamic spatial filter includes a weight matrix.

In some embodiments, the dynamic spatial filter includes a bias vector.

In some embodiments, the method can also involve visualizing the dynamic spatial filter in real-time at an interface.

In some embodiments, visualizing the dynamic spatial filter in real-time can include indicating a relative significance of at least one channel of the plurality of channels using one or more visual elements of the interface.

In some embodiments, the method involves visualizing the dynamic spatial filter in real-time by indicating signal quality feedback based in part on the learning task using one or more visual elements of the interface.

In some embodiments, the method further involves identifying an optimal location for hardware corresponding to at least one channel of the plurality of channels based in part on the dynamic spatial filter.

In some embodiments, the optimal location is determined in part by expected signals from an intended target of the at least one channel of the plurality of channels.

In some embodiments, the plurality of channels include a plurality of bio-signal sensors and the dataset aggregates bio-signal data from the bio-signal sensors. The learning task can involve predicting a brain state based in part on the bio-signal data, and the intended target can be a brain structure of a user the method involves performing measurements for the bio-signal data using a plurality of bio-signal sensors of the channels.

In some embodiments, the predicting a dynamic spatial filter from the representation of the dataset or the plurality channels using a neural network involves soft-thresholding channels of the plurality channels.

In some embodiments, the method further involves identifying a source space using the

dynamic spatial filter.

In some embodiments, the applying the dynamic spatial filter involves adjusting the channels to a form acceptable by the second neural network in the performing a learning task.

In some embodiments, the method further involves selectively transmitting at least one channel of the plurality of channels based in part on the dynamic spatial filter.

In some embodiments, the applying the dynamic spatial filter involves selectively transmitting at least one dynamically reweighed channel.

In some embodiments, the learning task involves predicting a sleep stage.

In some embodiments, the learning task involves detecting pathologies.

In some embodiments, the plurality of channels includes a plurality of bio-signal sensors,

and the dataset aggregates bio-signal data from the bio-signal sensors. The learning task can involve predicting a brain state based in part on the bio-signal data.

In some embodiments, the method can further involves adding noise or channel corruption to the dataset or the plurality of channels prior to the extracting a representation of the dataset or the plurality of channels.

In some embodiments, the noise or channel corruption can include at least one of additive white noise, spatially uncorrelated additive white noise, pink noise, simulated structured noise, and real noise.

In one aspect, embodiments described herein provide a system is disclosed herein for dynamically reweighing a plurality of channels according to relevance given a learning task or channel corruption using a neural network. The system has a plurality of channels for receiving data. The channels can have sensors or electrodes for capturing bio-signal data, for example. Each channel of the plurality of channels has data. The system has a computing device to receive data from the channels. The computing device can aggregate data from the multiple channels to generate a dataset, for example. The computing device can receive a dataset from the plurality of channels, extract a representation of the dataset or the channels, predict a dynamic spatial filter from the representation of the dataset or plurality of channels using neural network, apply the dynamic spatial filter to dynamically reweigh each of the channels, and perform a learning task using the reweighed channels and a neural network.

In some embodiments, the dynamic spatial filter comprises unbounded weights to preserve the conceptual connection between channel recombination and spatial filtering.

In some embodiments, neural network is trained for a predictive task.

In some embodiments, neural network is trained to perform a related learning task.

In some embodiments, the representation extracted from the dataset or plurality of channels comprises a first, second, third, or fourth order representation.

In some embodiments, the representation comprises a second order representation that comprises at least one of spatial covariance information, correlational information, and cosine similarity to capture dependencies between the plurality of channels.

In some embodiments, applying the dynamic spatial filter involves applying the dynamic spatial filter to input of a first layer of the second neural network.

In some embodiments, applying the dynamic spatial filter involves applying the dynamic spatial filter to output of layer of the neural network.

In some embodiments, the representation is at least one of non-linear relational data between the channels such as fractal representations, mutual information, and Granger causality.

In some embodiments, the channels have a plurality of sensors and the computing device can perform measurements for the dataset using the plurality of sensors of the channels.

In some embodiments, the channels receive data from a plurality of subdivisions of a sensor.

In some embodiments, the dataset involves data that is output of layer of the neural network and the performing a learning task using the reweighed channels and the neural network involves providing the reweighed channels to at least one subsequent layer to the layer of neural network.

In some embodiments, the channels include bio-signal, EEG, EMG, fNIRS, audio-signal, seismographic, radar, telescopic, motion data, chemical sensor data, protein sensor data, and video-signal.

In some embodiments, the channels can include an array of sensors and the computing device can perform measurements for the dataset using the array of sensors of the channels.

In some embodiments, the dynamic spatial filter includes a weight matrix.

In some embodiments, the dynamic spatial filter includes a bias vector.

In some embodiments, computing device further has a display. In some embodiments, the dynamic spatial filter can be visualized in real-time on the display.

In some embodiments, the interface can visualize the dynamic spatial filter in real-time using visual elements indicating a relative significance of at least one channel of the plurality of channels using one or more visual elements of the display.

In some embodiments, the interface can visualize the dynamic spatial filter in real-time using visual elements indicating signal quality feedback based in part on the learning task using one or more visual elements of the display.

In some embodiments, computing device can be further configured to identify an optimal location for hardware corresponding to at least one channel of plurality of channels based in part on the dynamic spatial filter.

In some embodiments, the optimal location is determined by the computing device in part by expected signals from an intended target of the at least one channel of the plurality of channels.

In some embodiments, the plurality of channels include a plurality of bio-signal sensors, and the dataset aggregates bio-signal data from the bio-signal sensors. The learning task can involve predicting a brain state based in part on the bio-signal data, and the intended target can be a brain structure of a user and the computing device can perform measurements for the bio-signal data using a plurality of bio-signal sensors of the channels.

In some embodiments, the predicting a dynamic spatial filter from the representation of the dataset or the plurality channels using a neural network involves soft-thresholding channels of the plurality channels.

In some embodiments, computing device is further configured to identify a source space using the dynamic spatial filter.

In some embodiments, the system can use the results of the learning task to adjust at least one trainable parameter of at least one of the neural networks.

In some embodiments, applying the dynamic spatial filter involves adjusting the channels to a form acceptable by the second neural network in the performing a learning task.

In some embodiments, the system is further configured to selectively transmit at least one channel of the plurality of channels based in part on the dynamic spatial filter.

In some embodiments, applying the dynamic spatial filter comprises selectively transmitting at least one dynamically reweighed channel.

In some embodiments, the learning task involves predicting a sleep stage.

In some embodiments, the learning task involves detecting pathologies.

In some embodiments, the plurality of channels includes a plurality of bio-signal sensors,

and the dataset aggregates bio-signal data from the bio-signal sensors. The learning task can involve predicting a brain state based in part on the bio-signal data.

In another aspect, embodiments described herein provide a system for dynamically reweighing a plurality of channels according to relevance given a learning task or channel corruption using a neural network. The system comprising a memory, and a processor coupled to the memory programmed with executable instructions. The instructions including a measuring component for measuring and collecting the datasets using a plurality of sensors for and transmitting the collected datasets to the interface using a transmitter, an interface for receiving a dataset from a plurality of channels; and a reweighing component for extracting a representation of the dataset or the plurality of channels, predicting a dynamic spatial filter from the representation of the dataset or the plurality of channels using a neural network, applying the dynamic spatial filter to dynamically reweigh each of the channels of the plurality channels, and performing a learning task using the reweighed channels and a second neural network.

### DESCRIPTION OF THE FIGURES

In the figures,

FIG. 1 illustrates a visual description of the Dynamic Spatial Filtering (DSF) component, according to some embodiments;

FIG. 2 illustrates a high-level schematic diagram of an implementation of a dynamic spatial filter, according to some embodiments;

FIG. 3 illustrates a high-level schematic diagram of an implementation of a dynamic spatial filter component within another neural network, according to some embodiments;

FIG. 4 illustrates a high-level schematic diagram of a dynamic spatial filter component, according to some embodiments;

FIG. 5 illustrates a high-level schematic diagram of an example learning system for a dynamic spatial filter, according to some embodiments;

FIG. 6 illustrates a flowchart example of operations using dynamic spatial filtering methods, according to some embodiments;

FIG. 7 illustrates a flowchart example of operations of a method of training a dynamic spatial filter component, according to some embodiments;

FIG. 8A illustrates neural network architectures f_{θ} used in pathology detection, according to some embodiments;

FIG. 8B illustrates neural network architectures f_{θ} used in sleep staging experiments, according to some embodiments;

FIG. 9A illustrates the corruption percentage of the 98 recordings of the Internal Sleep Dataset, according to some embodiments;

FIG. 10A illustrates the impact of noise strength on pathology detection performance of standard models where the η noise strength parameter was varied given a constant channel corruption probability of 50%, according to some embodiments;

FIG. 10B illustrates the impact of noise strength on pathology detection performance of standard models where the number of corrupted channels was varied given a constant noise strength of 1, according to some embodiments;

FIG. 10C illustrates the placement of 2 channels, 6 channels, and 21 channels, on the scalp of a user, according to come embodiments;

FIG. 11A illustrates the impact of noise strength on pathology detection performance for models coupled with no denoising strategy, Autoreject, and data augmentation where the η noise strength parameter was varied given a constant channel corruption probability of 50%, according to some embodiments;

FIG. 11B illustrates the impact of noise strength on pathology detection performance for models coupled with no denoising strategy, Autoreject, and data augmentation where the number of corrupted channels was varied given a constant noise strength of 1, according to some embodiments;

FIG. 12A illustrates the impact of noise strength on sleep staging performance for models coupled with no denoising strategy, Autoreject, and data augmentation where the η noise strength parameter was varied given a constant channel corruption probability of 50%, according to some embodiments;

FIG. 12B illustrates the impact of noise strength on sleep staging performance for models coupled with no denoising strategy, Autoreject, and data augmentation where the number of corrupted channels was varied given a constant noise strength of 1, according to some embodiments;

FIG. 13 illustrates recording-wise sleep staging results on ISD, according to some embodiments, according to some embodiments;

FIG. 14A illustrates the recording-wise sleep staging results on ISD showing test balanced accuracy for models coupled with (1) no denoising strategy, (2) Autoreject and (3) data augmentation, according to some embodiments;

FIG. 14B illustrates the recording-wise sleep staging results on ISD showing that good performance is obtained by combining data augmentation with DSF with logm(cov) and soft-thresholding (DSFm-st), according to some embodiments;

FIG. 14C illustrates the performance of the baseline models combined with the different

noise handling methodologies, according to some embodiments;

FIG. 15A illustrates the corruption process carried out to investigate the effective channel importance and spatial filters predicted by the DSF module trained on pathology detection, according to some embodiments;

FIG. 15B illustrates the distribution of channel contribution values ϕ using density estimate and box plots obtained when investigating the effective channel importance and spatial filters predicted by the DSF module trained on pathology detection, according to some embodiments;

FIG. 15C illustrates a subset of the spatial filters (median across all windows) plotted as topomaps for the three scenarios, according to some embodiments;

FIG. 16 illustrates normalized effective channel importance \hat{ϕ} predicted by the DSF module on two ISD sessions with naturally-occurring channel corruption (corruption throughout, and intermittent corruption), according to some embodiments;

FIG. 17A and FIG. 17B each illustrate a set of the performance of different attention module architectures on the TUAB evaluation set under increasing channel corruption noise strength, according to come embodiments; and

FIG. 18 is a schematic diagram of a computing device that implements the learning system of any of FIG. 2, FIG. 3, FIG. 4, and FIG. 5, in accordance with an embodiment.

### DETAILED DESCRIPTION

Machine learning processes and encoded models can be used for electroencephalography (EEG) monitoring. Machine learning models used for different applications (e.g., EEG monitoring) need to be robust to noisy data and randomly missing channels, especially when working with sparse montages (e.g., mobile EEG devices). However, deep neural networks trained end-to-end are not tested for robustness to corruption, especially to randomly missing channels. There can be challenges when sparse montages are used and limited computing power is available.

Lab and clinical applications can be translated to settings such as at-home or in remote locations. However, in these new settings, the number of sensors available is often limited and signal quality is harder to control. Moreover, the increasing availability of such devices (e.g., at-home or mobile) means the amount of data generated far exceeds the capacity of human experts (e.g., neurologists, sleep technicians, etc.) to analyze and manually annotate recordings, as traditionally done in research and clinical settings. Therefore, to expand the reach of these applications, there exists a need for automated, robust machine learning pipelines that can work with sparse montages or in challenging signal quality conditions.

EEG monitoring using channels of data from sensors or electrodes (including, for example, outside of a laboratory setting) can require machine learning models that are robust to noisy data and randomly missing channels, especially when working with sparse montages such as those of, for example, consumer-grade mobile EEG devices (e.g., those with 1-6 channels).

However, classical machine learning models and deep neural networks trained end-to-end on EEG are typically not tested for robustness to corruption, especially to randomly missing channels. Some approaches to use data with missing channels are not applicable or desirable when sparse montages are used and limited computing power is available.

To alleviate this problem, embodiments described herein provide dynamic spatial filtering (DSF), which can involve a multi-head attention module that can be plugged before the first layer of a neural network (or subsequent layer of the neural network) to handle missing EEG channels by learning to focus on good channels and ignore bad ones. DSF was tested on public EEG data encompassing ˜4,000 recordings with simulated channel corruption and on a private dataset of ˜100 at-home recordings of mobile EEGs with natural corruption. The proposed approach can reach the same performance as baseline models when no noise is applied but can outperform baselines by as much as 29.4% accuracy when strong channel corruption is present. Moreover, DSF outputs can be interpretable, making it a useful tool also for monitoring effective channel importance and signal quality in real-time. This approach may enable analysis of EEG in challenging settings where channel corruption hampers brain signal readings.

Some embodiments described herein provide a method to handle corrupted channels in sparse EEG data.

Some embodiments described herein provide an attention mechanism neural network architecture that can dynamically reweigh EEG channels according to their relevance given a predictive task.

EEG monitoring can enable low-cost brain function and health applications, such as neuro-modulation, stimulation, sleep monitoring, sleep intervention, pathology screening, neurofeedback therapy, brain-computer interfacing and anaesthesia monitoring. EEG monitoring can also be used to enable low-cost entertainment applications, such as applications in music, movies and shows, and gaming. It can also enable electromyographic applications such as in the field of prosthetics. Mobile EEG applications can be translated from lab and clinic settings to non-traditional settings such as at-home, ambulatory assessment, or in remote locations. The EEG applications can make brain health monitoring more accessible in real-world settings. However, in these new settings, the number of electrodes or channels available can often be limited and signal quality can be harder to control. Moreover, the increasing availability of these devices means the amount of data generated can exceed the capacity of human experts (e.g., neurologists, sleep technicians, etc.) to analyze and manually annotate recordings, as traditionally done in research and clinical settings. Some embodiments described herein can provide EEG applications, and automated, robust machine learning pipelines that work with sparse montages and in challenging signal quality conditions. Novel methods facilitating clinical and research applications in real-world settings, especially with sparse EEG montages are therefore needed.

EEG prediction pipelines can be benchmarked on datasets recorded in well-controlled conditions that are mostly clean when compared to data from mobile EEG. As a consequence, it can be unclear how methods designed for laboratory data will cope with signals encountered in real-world contexts or how robust to noise these methods are. This can be especially critical for mobile EEG recordings that may contain a varying number of usable channels as well as overall noisier signals, in contrast to most research- and clinical-grade recordings. In addition, the difference in number of channels between research and mobile settings also means that interpolating bad channels offline (as can be done in recordings with dense electrode montages) is likely to fail on mobile EEG devices given their more limited spatial information. Adding to the challenge, signal quality and/or quality of EEG data may not be static but can vary extensively inside a recording, meaning predictive models should handle noise dynamically. Not only should machine learning pipelines produce predictions that are robust to (changing) sources of noise in EEG, but they can also do so in a way that is transparent or interpretable. For instance, if noise is easily identifiable, corrective action can be quickly taken by experimenters or users during a recording.

Not all sources of noise can affect EEG recordings in the same way. Physiological artifacts are large electrical signals that are generated by current sources outside the brain such as heart activity, eye or tongue movement, muscle contraction, sweating, etc. Depending on the EEG electrode montages and the setting of the recording (e.g., eyes open or closed), these artifacts can be more or less disruptive to measuring the brain activity of interest. Movement artifacts, on the other hand, are caused by the relative displacement of EEG electrodes with respect to the scalp, and can introduce noise of varying spectral content or create sharp deflections in the affected electrodes during the movement. If an electrode cannot properly connect with the skin (e.g., after a movement artifact or because it was not correctly set up initially), its reading can contain little or no physiological information and instead pick up on instrumentation and/or environmental noise (e.g., noise introduced in the electronics circuit or powerful electromagnetic sources present around the recording equipment). These are commonly referred to as “bad” or “missing” channels. Hereinafter, these channels will be referred to as “corrupted channels” to explicitly include the case where a signal corruption mechanism (e.g., active noise sources in uncontrolled environments) must be accounted for by predictive models. While channel corruption affects EEG recordings in all contexts, it is more likely in real-world mobile EEG recordings than in controlled laboratory settings where trained experimenters can monitor and remedy bad electrodes during the recording. Moreover, as opposed to controlled experiments where dense electrode montages can allow interpolating corrupted channels offline, the limited spatial information of some mobile EEG devices makes this approach much more challenging. As a result, there exists as a need for EEG applications for handling the specific problem of channel corruption in sparse montages of, for example, mobile EEG settings.

Embodiments described herein provide an attention mechanism component to handle corrupted channel data, which can be based on the concept of “scaling attention”. In some embodiments, this component can be inserted before the first layer of any convolutional neural network architecture in which activations have a spatial dimension and can be trained end-to-end for the prediction task at hand. In some embodiments, this module can be inserted between any other layers of a convolution neural network to reweigh channel data based on relevance to a predictive task.

Disclosed herein are systems, methods, and devices directed at training or using dynamic spatial filters (DSF) to reweigh channels according to their corruption or the learning or predictive task.

### Notation

Throughout the description, [symbol] q [symbol] is denoted as the set {1, . . . , q}. The index t refers to time indices in the multivariate time series S∈ [symbol] ^{C×M}, where M is the number of time samples and C is the number of EEG channels. S is further divided into non-overlapping windows X∈ [symbol] ^{C×T} where T is the number of time samples in the window. y∈ [symbol] denotes the target used in the learning task. Typically, [symbol] is [symbol] L [symbol] for a classification problem with L classes.

### Approaches to Noise-Robust EEG Processing

Strategies for dealing with noisy data can be divided into three categories (Table 1): (1) ignoring or rejecting noisy segments, (2) implicit denoising, i.e., methods that allow models to work despite noise, and (3) explicit denoising, i.e., methods that rely on a separate preprocessing step to handle noise or missing channels before inference.

TABLE 1
illustrates methods for dealing with noisy EEG data.

| | Approach | Examples | Notes |
|---|---|---|---|
| Ignore or reject noise | No denoising | | Might not work in real- |
| | | | life applications (out |
| | | | of the lab/clinic) |
| | Removing bad | | Doesn't allow online |
| | epochs | | predictions; Might |
| | | | discard useful |
| | | | information |
| Implicit denoising | Robust input | Covariance matrices | Might not work if too |
| | representations | in Riemannian | few channels |
| | | tangent space | available |
| | | Topomaps | Expensive |
| | | | preprocessing step; |
| | | | Might not work if too |
| | | | few channels |
| | | | available |
| | Robust signal | Lomb-Scargle | Only useful for |
| | processing | periodogram | missing samples, not |
| | techniques | | missing channels |
| | Robust machine | Handcrafted features | Requires feature |
| | learning classifiers | and random forest | engineering step |
| Explicit denoising | Spatial projection- | Signal Space | Might not work if too |
| | based approaches | Separation (SSS) for | few channels |
| | | MEG | available; Additional |
| | | ICA-based denoising | preprocessing step; |
| | | | Preprocessing might |
| | | | discard important |
| | | | information for |
| | | | learning task |
| | Automated correction | Autoreject, FASTER, | Expensive |
| | | PREP | preprocessing step |
| | Model-based | Deep learning-based | Separate training |
| | interpolation/ | superresolution (GAN, | step; Additional |
| | reconstruction | LSTM, AE, etc.) | inference step to |
| | | | reconstruct at test |
| | | | time; Requires |
| | | | separate procedure to |
| | | | detect corrupted |
| | | | channels |
| | | Tensor | |
| | | decomposition, | |
| | | compressed sensing | |
| Interpretable | Channel corruption- | Dynamic Spatial | Trained end-to-end, |
| denoising | invariant architecture | Filtering (described | no additional |
| | | herein) | preprocessing, |
| | | | interpretable, works |
| | | | with sparse montages |

The simplest way to deal with noise in EEG is to assume that it is negligible or to simply discard bad segments. For instance, a manually selected amplitude or variance threshold or a classifier trained to recognize artifacts can be used to identify noisy segments to be ignored. This approach, though commonplace, can be ill-suited to mobile EEG settings where noise cannot be assumed to be negligible, and to online applications where model predictions may need to be continuously available. Moreover, this approach can discard windows due to a small fraction of bad electrodes, potentially losing usable information from other channels.

Implicit denoising approaches can be used to design noise-robust processing pipelines that do not contain a specific noise handling step. A first group of implicit denoising approaches uses representations of EEG data that can be robust to missing channels. For instance, multichannel EEG can be transformed into topographical maps (“topomaps”) which may be more robust or less sensitive to the absence of one or few channels. Moreover, by representing power in specific frequency bands as color channels, the model can learn to focus on the frequencies where signal-to-noise ratio (SNR) is better. This representation can then be fed into a standard convolutional neural network (ConvNet) architectures. This approach is likely to perform poorly on sparse montages (e.g., 4 channels) as spatial interpolation might fail if channels are missing and the absence of a channel can significantly impact the topographical maps. Moreover, this approach can require a computationally demanding preprocessing and feature extraction step, undesirable in online and low-computational resources contexts. Similarly, but in a traditional machine learning setting representing input windows as covariance matrices and using Riemannian geometry-aware models may not require common noise correction steps to reach high performance. It is unclear though how this approach could be integrated in neural network architectures and how it would perform on sparse montages. Integration of this approach into neural network architectures is not straightforward. Signal processing techniques can also promote invariance to certain types of noise. For instance, the Lomb-Scargle periodogram can be used to extract spectral representations that are robust to missing samples. However, this approach may fail if channels are completely missing. Finally, implicit denoising can be achieved with traditional machine learning models that are inherently robust to noise. For instance, random forests trained on handcrafted EEG features can be notably more robust to low SNR inputs than univariate models.

Although promising, this approach may be limited by its feature engineering step, as features (1) rely heavily on domain knowledge, (2) might not be optimal to the task, and (3) require an additional processing step which can be prohibitive in limited resource contexts.

Explicit noise handling can be implemented by automatically correcting corrupted signals or predicting missing or additional channels from the available ones. Spatial projection approaches aim at projecting the input signals to a noise-free subspace before projecting the signals back into channel-space, e.g., using independent component analysis (ICA) or principal components analysis (PCA). While approaches such as ICA are powerful tools to mitigate artifact and noise components in a semi-automated way, their efficacy diminishes when only few channels are available. For example, in addition to introducing an additional preprocessing step, these approaches may be ill-suited to sparse montages. Also, because explicit denoising can be decoupled from the learning task, it is unclear how much discriminative information will be discarded during the preprocessing. The fact that preprocessing can be done independently from the supervised learning task or the statistical testing procedure actually makes the selection of preprocessing parameters (e.g., number of good components) challenging. Fully automated denoising pipelines exist. For instance, some methods combine artifact correction, noise removal and bad channel interpolation into a single automated pipeline. Autoreject is another pipeline that uses cross-validation to automatically select amplitude thresholds to use for rejecting windows and flagging bad channels to be interpolated window-wise. These approaches can be well-suited to offline analyses where the morphology of the signals is of interest, however they can be typically computationally intensive and can also be decoupled from the statistical modeling. Additionally, it unclear how interpolation can be applied when using bipolar montages (i.e., that do not share a single reference), as is often the case in e.g., polysomnography and epilepsy monitoring.

Generic machine learning models may recover bad channels. For instance, generative adversarial networks (GANs) can be trained to recover dense EEG montages from a few electrodes. Other similar methods have been proposed, e.g., using a long short-term memory (LSTM) neural network, an autoencoder, or tensor decomposition and compressed sensing. However, these methods postulate that the identity of corrupted channels is known ahead of time, which is a non-trivial assumption in practice.

In some embodiments, an interpretable end-to-end denoising approach can learn implicitly to work with corrupted sparse EEG data and does not require additional preprocessing steps.

### Dynamic Spatial Filtering: Second-Order Attention for Learning on Noisy EEG Signals

A goal behind dynamic spatial filtering (DSF) is to help a neural network focus on the most important channels, at each time instant, given a specific machine learning task on EEG. To do so, a spatial attention mechanism can be introduced that can dynamically re-weigh channels according to their predictive power. This idea is inspired by attention mechanisms, most specifically the “scaling attention” approach proposed in computer vision. Notably, DSF can leverage second-order information, i.e., spatial covariance, to capture dependencies between EEG channels.

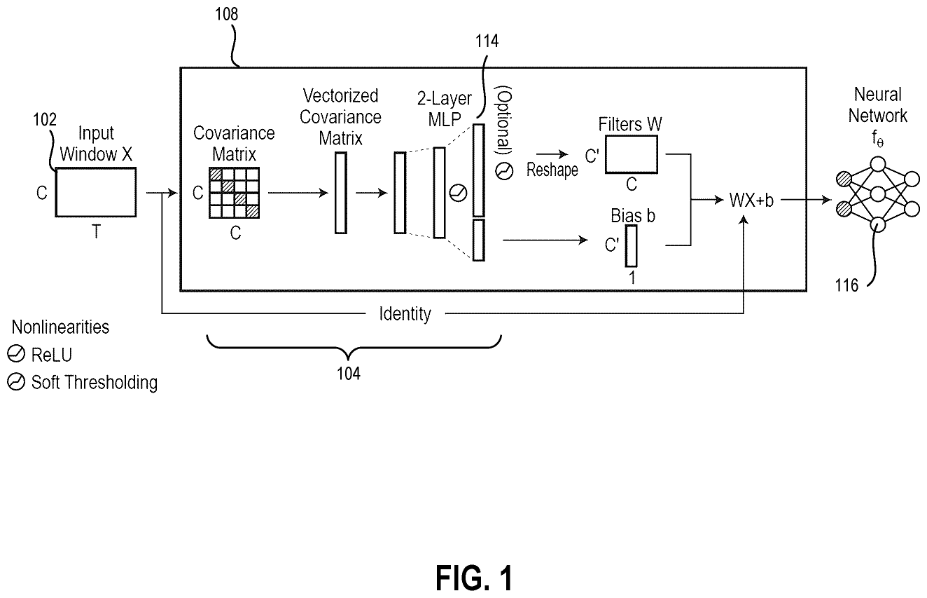

FIG. 1 illustrates a visual description of the Dynamic Spatial Filtering (DSF) component, according to some embodiments. An input X with C spatial channels is processed by a 2-layer MLP to produce a set of C′ spatial filters W and biases b that dynamically transform the input X (104). This allows the following layers of a neural network to ignore corrupted channels and focus on the most informative ones.

By way of example, dynamic spatial filter component 108 can use a neural network 114 to dynamically reweigh a plurality of channels 102. A neural network 116 can use the reweighed channels to perform a learning task.

Experimental, non-limiting results described below were performed in the supervised classification setting. A model f_{θ}: X→ [symbol] with parameters θ (e.g., a convolutional neural network) is trained to predict the class y of EEG windows X. For this, f_{θ} is trained to minimize the loss [symbol] , e.g., the categorical cross-entropy loss, over the example label pairs (Xᵢ, yᵢ):

$${f}_{θ} = \begin{matrix}argmin \\ θ\end{matrix} [ ℒ ( {f}_{θ} ( {X}_{i} ) , {i}_{} ) ] \qquad ( 1 )$$

The performance of f_{θ} when random channels are corrupted is of interest and more specifically when channel corruption occurs at test time (i.e., when training data is mostly clean). Toward this goal, an attention-based module m_{DSF}: [symbol] ^{C×T}→ [symbol] ^{C′×T} can be inserted into f_{θ} which performs a (fixed) transformation Φ(X) to extract relevant spatial information from X, followed by a re-weighting mechanism for the input signals.

In order to implicitly handle noise in neural network architectures, an attention module where second-order information is extracted from the input was designed and used to predict weights of a linear transformation of the input EEG channels, that are optimized for the learning task (FIG. 1). Applying such linear transforms to multivariate EEG signals is commonly referred to as “spatial filtering”. This way, the model can learn to ignore noisy outputs and/or to re-weigh them, while still leveraging any remaining spatial information. This module can be applied to the raw input X as set out below.

The dynamic spatial filter (DSF) module m_{DSF} can be defined as:

$${m}_{DSF} ( X ) = {W}_{DSF} ( X ) X + {b}_{DSF} ( X ) \qquad ( 2 )$$

where W_{DSF}∈ [symbol] ^{C′×C} and b_{DSF}∈ [symbol] ^{C′} are obtained by reshaping the output of a neural network, e.g., a multilayer perceptron (MLP), h_{θ}_{_{DSF}}(Φ(X))∈ [symbol] ^{C′+(C+1)} (see FIG. 1). Under this formulation, each row in W_{DSF} corresponds to a spatial filter that linearly transforms the input signals into another virtual channel. Here, C′ can be set to the number of input spatial channels C or considered as a hyperparameter of the attention module (in which case it can be used to increase the diversity of input channels in models trained on sparse montages (C′>C) or perform dimensionality reduction to reduce computational complexity (C′<C)). When C′=C, if the diagonal of W_{DSF} is 0, W_{DSF} corresponds to a linear interpolation of each channel based on the C−1 others. Heavily corrupted channels can be ignored by giving them a weight of 0 in W_{DSF}. To facilitate this behavior, a soft-thresholding element-wise nonlinearity can be applied to W_{DSF}:

$${W}_{DSF}^{′} = sign ( {W}_{DSF} ) max ( \vert {W}_{DSF} \vert - τ , 0 ) , \qquad ( 3 )$$

where τ is a threshold empirically set to 0.1 and |⋅| is the element-wise absolute value and both the sign and max operators are applied element-wise. The spatial information extracted by the transforms Φ(X) can be, for example, (1) the log-variance of each input channel or (2) the flattened upper triangular part of the matrix logarithm of the covariance matrix of X (In practice, if a channel is “flat-lining” (has only 0s) inside a window and therefore has a variance of 0, its log-variance is replaced by 0. Similarly, if a covariance matrix eigenvalue is 0 when computing the matrix logarithm (see Equation 5), its logarithm is replaced by 0.).

When reporting results, models are denoted as DSFd and DSFm when DSF take the log-variance or the matrix logarithm of the covariance matrix, respectively, and the suffix “-st” is added to indicate the use of the soft-thresholding nonlinearity, e.g., DSFm-st.

Interestingly, the DSF component can be seen as a multi-head attention mechanism with real-valued attention weights and where each head is tasked with producing a linear combination of the input spatial signals.

Finally, the attention given by DSF to each input channel can be inspected by computing the “effective channel importance” metric ϕ∈ [symbol] ^{C} where ϕⱼ=\sqrt{Σᵢ₌₁^{C′}Wᵢⱼ²}. Intuitively, ϕ measures 7 how much each input channel is used by m_{DSF} to produce the output virtual channels. A normalized version:

$$ϕ = \frac{ϕ}{{max}_{i} {ϕ}_{i}} \qquad ( 4 )$$

can also be used to obtain a value between 0 and 1. This straightforward way of inspecting the functioning of the DSF component can facilitate the identification of important or noisy channels.

“Effective channel importance” measures how useful the actual data of a channel is. It is not to be confused with the theoretical importance of a channel, i.e., the fact that in theory some channels (given good signal quality) might be more useful for some tasks than other channels.

Therefore, in this work, when discussing the “importance” of a channel, reference is made to the usefulness of the actual signal collected with that channel with respect to the task. For instance, a corrupted channel will likely have low “importance”, although the neurophysiological information available at that location would be useful should the channel not be corrupted. The use of the word importance in the present context is in line with statistical machine learning referring to “feature importance” as quantified for example using “permutation importance”.

To further help the models in the experimental settings, non-limiting embodiments learn to be robust to noise, a data augmentation procedure that randomly corrupts channels was designed. Specifically, channel corruption is simulated by performing a masked channel-wise convex combination of input channels and Gaussian white noise Z∈ [symbol] ^{C×T}:

$$\tilde{X} = ( 1 - η ) diag ( v ) X + η diag ( v ) Z + diag ( 1 - v ) X , \qquad ( 5 )$$

where Zᵢⱼ˜ [symbol] (0, σₙ²) for i∈ [symbol] T [symbol] and j∈ [symbol] C [symbol] , η∈[0, 1] controls the relative strength of the noise, and v∈{0, 1}^{C} is a masking vector that controls which channels are corrupted. The operator diag(x) creates a square matrix filled with zeros whose diagonal is the vector x. Here, v is sampled from a multinouilli distribution with parameter p. Each window X is individually corrupted using random parameters σₙ˜ [symbol] (20,50) μV, η˜ [symbol] (0.5, 1), and a fixed p of 0.5.

### From Simple Interpolation to Dynamic Spatial Filtering

DSF is conceptually linked to, but different from, noise handling pipelines such as Autoreject which rely on an interpolation step to reconstruct channels that have been identified as bad or corrupted. Specifically, these pipelines use head geometry-informed interpolation methods (based on the 3D coordinates of EEG electrodes and spline interpolation) to compute the weights necessary to interpolate each channel using a linear combination of the C−1 other channels. From this perspective, a naive method of handling corrupted channels might be to always replace each input EEG channel by its interpolated version based on the other C−1 channels. An “interpolation-only” module mᵢₙₜₑᵣₚ could be written as:

mᵢₙₜₑᵣₚ(X)=WᵢₙₜₑᵣₚX, (6)

where Wᵢₙₜₑᵣₚ is a C×C real-valued matrix with a 0-diagonal (Wᵢₙₜₑᵣₚ can be set or initialized using head geometry information or can be learned from the data end-to-end). The limitation of this approach is that given at least one corrupted channel in the input X, the interpolated version of all non-corrupted channels will be reconstructed in part from corrupted channels. This means noise may still be present, however given enough clean channels, its impact might be mitigated.

Improving upon the naive interpolation-only approach, the model can decide whether (and to what extent) channels should be replaced by their interpolated version. For instance, if the channels in a given window are mostly clean, it might be desirable to keep the initial channels; however, if the window is overall corrupted, it might instead be better to replace channels with their interpolated version. This leads to a “scalar-attention” module m_{scalar}:

$$\\ \begin{matrix}{m}_{scalar} ( X ) = diag ( {α}_{X} ) X + ( 1 - diag ( {α}_{X} ) ) WX , & ( 7 )\end{matrix}$$

where α_{X}∈[0, 1]^{C} is the attention weight predicted by an MLP conditioned on X (e.g., on its covariance matrix) and W is the same as for the interpolation-only module. While this approach is more flexible, it still suffers from the same limitation as before: there is a chance interpolated channels will be reconstructed from noisy channels. Moreover, the fact that the attention weight is applied globally, i.e., a single weight applies to all C channels, limits the ability of the module to focus on reconstructing corrupted channels only.

Instead, the “vector attention” module m_{vector} introduces channel-wise attention weights, so that the interpolation can be independently controlled for each channel:

$${m}_{vector} ( X ) = diag ( {α}_{X} ) X + ( I - diag ( {α}_{X} ) ) WX \qquad ( 8 )$$

where α_{X}∈[0, 1]C is again obtained with an MLP and W is as above. Although more flexible, this version of the attention module still faces the same problem caused by static interpolation weights.

To solve this issue, the previous approach can be expanded by both predicting an attention vector ax as before and dynamically interpolating with a matrix W_{X}∈ [symbol] ^{C×C} (with a 0-diagonal) predicted by another MLP:

$${m}_{dynamic} ( X ) = diag ( {α}_{X} ) X + ( I - diag ( {α}_{X} ) ) {W}_{X} X , \qquad ( 9 )$$

In practice, a single MLP can output C×C real values, which can then be reshaped into a 0-diagonal interpolation matrix W and a C-length vector whose values are passed through a sigmoid nonlinearity to obtain the attention weights α_{X}. An interesting property of this formulation which holds for m_{vector} too is that α_{X} can be directly interpreted as the level to which each channel is replaced by its interpolated version. However, in contrast to m_{vector} the interpolation filters can dynamically adapt to focus on the most informative channels.

Finally, Equation 9 can be rewritten as a single matrix product:

$${m}_{dynamic} ( X ) = ( diag ( {α}_{X} ) + ( I - diag ( {α}_{X} ) ) {W}_{X} ) X = {Ω}_{X} X , \qquad ( 10 )$$

where, denoting the element i, j of matrix W_{X} as Wᵢⱼ,

$${Ω}_{X} = [ \begin{matrix}{α}_{1} & ( 1 - {α}_{1} ) {W}_{12} & ⋯ & ( 1 - {α}_{1} ) {W}_{1 C} \\ ( 1 - {α}_{2} ) {W}_{21} & {α}_{2} & ⋯ & ( 1 - {α}_{2} ) {W}_{2 C} \\ ⋮ & ⋮ & ⋱ & ⋮ \\ ( 1 - {α}_{C} ) {W}_{C 1} & ( 1 - {α}_{C} ) {W}_{C 2} & ⋯ & {α}_{C}\end{matrix} ] , \qquad ( 11 )$$

The matrix Ω_{X} contains C² free variables, that are all conditioned on X through an MLP. The constraints on Ω_{X} are then relaxed to obtain a simple matrix W_{DSF} where there are no dependencies between the parameters of a row and the diagonal elements are allowed to be real-valued. This new unconstrained formulation can be interpreted as a set of spatial filters that perform linear combinations of the input EEG channels. An additional bias term can be introduced to recover the DSF formulation:

$${m}_{D S F} ( X ) = {W}_{D S F} ( X ) X + {b}_{D S F} ( X ) , \qquad ( 12 )$$

This bias term can be interpreted as a dynamic re-referencing of the virtual channels. In contrast to the interpolation-based formulations, DSF allows controlling the number of “virtual channels” C′ to be used in the downstream neural network in a straightforward manner (e.g., enabling the use of montage-specific DSF heads that could all be plugged into the same f_{θ} with fixed input shape). In some embodiments, neural networks trained with DSF can outperform interpolation-based formulations.

### Representations of Spatial Information in the DSF Module

Given some EEG signals X∈ [symbol] ^{C×T}, where T is the number of times samples in X, and which we assume to be zero-mean, an unbiased estimate of their covariance reads:

$$\sum ( X ) = \frac{{XX}^{T}}{T} ∈ {ℝ}^{CxC} \qquad ( 13 )$$

The zero-mean assumption is justified after some high-pass filtering or simple baseline correction of the signals. To assess whether one channel is noisy or not, a human expert annotator might rely on the power of a signal and its similarity with the neighbouring channels to decide whether it is noisy or not. This information can be encoded in the covariance matrix.

Multiple signal processing techniques rely on estimating Σ. For instance, common spatial patterns (CSP) performs generalized eigenvalue decomposition of covariance matrices to identify optimal spatial filters for maximizing the difference between two classes. Riemannian geometry approaches to EEG classification and regression rather leverage the geometry of space of symmetric positive definite (SPD) matrices to develop geometry-aware metrics. They can be used to average and compare covariance matrices, which can outperform other classical approaches. Artifact handling pipelines such as the Riemannian potato and Artifact Subspace Reconstruction (ASR) can further rely on covariance matrices to identify bad epochs or attenuate noise.

The values in a covariance matrix often follow a heavy-tailed distribution, making it challenging to use them directly in a neural network. Therefore, knowing that neural networks can be easier to train when the distribution of input values concentrated, it is helpful to standardize the covariance values before feeding them to the network. While scalar non-linear transformations (e.g., logarithms) could help reduce the range of values and facilitate the neural network's task, the geometry of SPD matrices actually calls for metrics that respect the Riemannian structure of the SPD matrices' Riemannian manifold. For instance, this means using the matrix logarithm instead of naively flattening the upper triangle and diagonal of the matrix. For an SPD matrix S, whose orthogonal eigendecomposition reads S=UΛU^{T}, where Λ=diag(λ₁, . . . , λₙ) contains its eigenvalues, the matrix logarithm log(S) is given by:

log(S)=U diag(log(λ₁), . . . , log(λₙ))U^{T} (14)

The diagonal and upper-triangular of log(S) can then be flattened into a vector with C(C+1)/2 values, which is then typically used with linear models, e.g., support vector machines (SVM) or logistic regression.

Other options to provide input values in a restricted range exist. For instance, the elementwise logarithm of the diagonal of the covariance matrix (i.e., the log-variance of the input signals) could be used if pairwise covariance information is deemed not critical to the role played by the neural network. Alternatively, Pearson's correlation matrix R could be used instead of the covariance matrix. R has the advantage that its values are already in a well-defined range (−1, 1), yet it is blind to channel variance.

It can be seen as the covariance matrix of the z-score normalized signals:

$$R ( X ) = \frac{\sum ( X )}{σ ⊗ {σ}^{T}} \qquad ( 15 )$$

where σ=\sqrt{diag}(Σ), i.e., the signal-wise standard deviation vector and is the outer product. However, the correlation matrix is also an SPD matrix, and should therefore be transformed with the matrix logarithm too. Finally, given that the diagonal of R is filled with 1s, a modified version R* can be designed where the diagonal of R is replaced with the log-variance computed from Σ.

Described herein, two spatial representations were focused on: the channel wise-variance obtained from the diagonal of Σ, and the matrix logarithm of Σ, though others are possible. Both may improve robustness on the pathology detection and sleep staging tasks.

### Implementing DSF

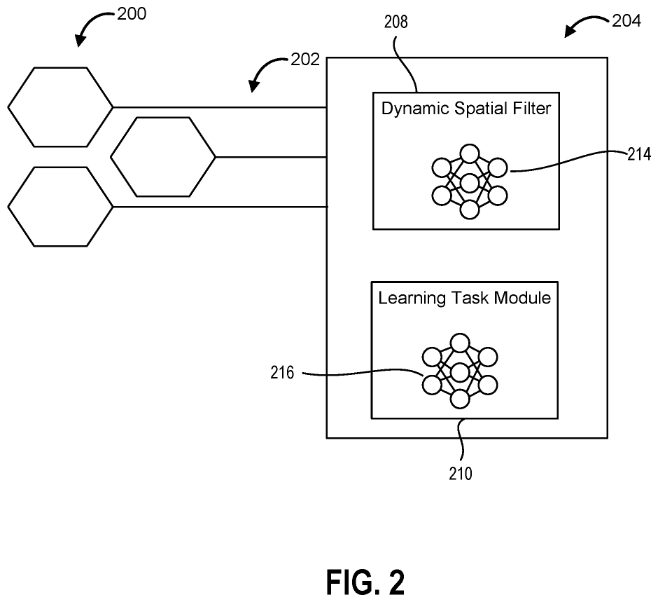

FIG. 2 illustrates a high-level schematic diagram of an implementation of a dynamic spatial filter, according to some embodiments.

FIG. 2 shows a computing device 204 receiving data from a plurality of channels 202 and comprising a dynamic spatial filter component 208 and a learning task module 210. Dynamic spatial filter component 208 can comprise neural network 214. Learning task module 210 can comprise neural network 216. Optionally, the plurality of channels 202 can receive data from a plurality of sensors 200.

For simplicity only one computing device 204 is shown but the system may include more computing devices and the components found within (i.e., dynamic spatial filter component 208 and learning task module 210) can be distributed amongst those computing devices. The computing device can have different hardware components that may be connected in various ways including directly coupled, indirectly coupled via a network, and distributed over a wide geographic area and connected via a network (which may be referred to as “cloud computing”).

For example, and without limitation, the computing device 204 may be a server, network appliance, set-top box, embedded device, computer expansion module, personal computer, laptop, personal data assistant, cellular telephone, smartphone device, UMPC tablets, video display terminal, gaming console, electronic reading device, and wireless hypermedia device or any other computing device capable of being configured to carry out the methods described herein. The computing device 204 can have a non-transitory memory storing executable code or instructions and a hardware processor. The computing device 204 can use its hardware processor to execute the code to generate or modify the dynamic spatial filter component 208 and learning task module 210. The computing device 204 can also have a transceiver to receive and transmit data. The computing device 204 can use its hardware processor to extract a representation of a dataset received from channels 202. The computing device 204 can store the extracted representation in its memory. The computing device 204 can use its hardware processor to predict the dynamic spatial filter 208 from the representation of the dataset or the channels 202 using a neural network 214. The computing device 204 can apply the dynamic spatial filter 208 to dynamically reweigh each of the channels 202. The computing device 204 can use its hardware processor to perform a learning task 210 using the reweighed channels and a second neural network 216. The computing device 204 can store the output results in its memory. Further details, variations, and implementations are provided herein.

In some embodiments, plurality of channels 202 can pass a dataset to dynamic spatial filter component 208 at a computing device 204. Dynamic spatial filter component 208 can use neural network 214 to extract a representation of the dataset or plurality of channels 202 and use that representation to predict a dynamic spatial filter from the dataset or plurality of channels 202.

Dynamic spatial filter component 208 can apply that dynamic spatial filter to reweigh the plurality of channels. Learning task module 210 can perform a learning task with the reweighed channels using neural network 216.

In one aspect, embodiments described herein provide a system is disclosed herein for dynamically reweighing a plurality of channels according to relevance given a learning task or channel corruption using a neural network. The system has a plurality of channels 202 for receiving data. The channels 202 can have sensors or electrodes for capturing bio-signal data, for example. Each channel of the plurality of channels has data. The system has a computing device 204 to receive data from the channels 202. The computing device 204 can aggregate data from the multiple channels 202 to generate a dataset, for example. The computing device 204 can receive a dataset from the plurality of channels 202, extract a representation of the dataset or the channels 202, predict a dynamic spatial filter from the representation of the dataset or plurality of channels 202 using neural network 214, apply the dynamic spatial filter to dynamically reweigh each of the channels 202, and perform a learning task using the reweighed channels and a neural network 216. In some embodiments, the representation is the result of one or more transformations that makes the dataset easier for the neural network to work with. In some embodiments, the system can perform measurements for the dataset using a plurality of sensors 200. In such embodiments, the each channel may receive the data from a corresponding sensor or the plurality of sensors.

For example, in some embodiments, the system may dynamically reweigh a plurality of EEG channels according to relevance given a learning task or channel corruption using a neural network. The system has a plurality of EEG channels 202 for receiving data. The channels 202 can have EEG sensors or electrodes for capturing bio-signal data, for example. Each channel of the plurality of channels has data. The system has a computing device 204 to receive data from the channels 202. The computing device 204 can aggregate data from the multiple channels 202 to generate a dataset, for example. The computing device 204 can be configured to receive a dataset from the plurality of channels 202, extract a representation of the dataset or the channels 202, predict a dynamic spatial filter from the representation of the dataset or plurality of channels 202 using neural network 214, apply the dynamic spatial filter to dynamically reweigh each of the channels 202, and perform a learning task using the reweighed channels and a neural network 216.

In some embodiments, the dynamic spatial filter comprises unbounded weights to preserve the conceptual connection between channel recombination and spatial filtering. In some embodiments, the dynamic spatial filter predicted by the system is not limited to a linear combination of channel weights. In some embodiments, the weights can be any real number as predicted by the system such that the predicted spatial filters more closely reflect which channels will contribute the most to the system performance.

In some embodiments, neural network 216 is trained for a predictive task. In some embodiments neural network 216 is trained to predict a result based on inputs. Predictive tasks can include tasks such as classification, regression, segmentation, and clustering.

In some embodiments, neural network 216 is trained to perform a related learning task. In some embodiments, neural network 216 is trained to learn new feature spaces. In some embodiments, neural network 216 is trained to create embeddings. Learning tasks can include tasks such as reinforcement learning, density estimation, reconstruction, and generative modelling.

In some embodiments, neural network 216 is used to perform a learning task by performing a predictive task or a related learning task. In such embodiments, the learning task may be completed by another decision-making entity (e.g., another neural network, a machine learning model, a rule-based algorithm, or a person) using the predictive task or the related learning task completed by neural network 216.

In some embodiments, the representation extracted from the dataset or plurality of channels 202 comprises a first, second, third, or fourth order representation. In some embodiments, the representation comprises spatial covariance, correlational information, or cosine similarity. In some embodiments, the representation captures dependencies between the channels. In some embodiments, the representation comprises a second order representation that comprises at least one of spatial covariance information, correlational information, and cosine similarity to capture dependencies between the plurality of channels. In some embodiments, a second order spatial covariance matrix can be extracted from the dataset or the plurality of channels and vectorized. Such representations can be passed into a neural network to predict a dynamic spatial filter, the neural network having been trained to do so to complete a learning task.

In some embodiments, the representation is at least one of non-linear relational data between the channels such as fractal representations, mutual information, and Granger causality.

In some embodiments, the channels 202 have a plurality of sensors 200 and the computing device can perform measurements for the dataset using the plurality of sensors 200 of the channels 202. The sensors 200 or electrodes can be placed at different positions on the user to capture bio-signal data of the user, for example. In some embodiments, each sensor 200 provides data to a corresponding channel in the plurality of channels 202. The computing device 204 receives the data generated by the sensors 200 from the plurality of channels 202. The sensors 200 can capture bio-signal data, such as brainwave or brain activity data, for example. The channels 202 can have different types of sensors 200. Example sensors 200 include at least one of bio-signal, EEG, EMG, fNIRS, audio-signal, seismographic, radar, telescopic, motion sensors, chemical sensor data, protein sensor data, and video-signal. The data can involve different types of data, such as bio-signal, EEG, EMG, fNIRS, audio-signal, seismographic, radar, telescopic, motion data, chemical sensor data, protein sensor data, and video-signal, for example.

In some embodiments, the channels 202 receive data from a plurality of subdivisions of a sensor. Subdivisions can include spatial subdivisions of the sensor, frequency subdivisions of a measured signal, or other divisions that can be used to assess the signal from the sensor. In such embodiments, the system can be configured to determine, for example, a second-order covariance matrix of the subdivisions and, using this, extract and apply a dynamic spatial filter, for example, to denoise the signal by reducing the weight of noisy regions of a signal based on the rest of the signal. In some embodiments, dynamic spatial filter component 208 can reweigh the plurality of channels to reduce noise or channel corruption in the sensor having the plurality of subdivisions of a sensor. In some embodiments, a dynamic spatial filter component 208 can apply a dynamic spatial filter to a plurality of subdivisions of a sensor for each sensor in a system which can denoise the sensors on a sensor-by-sensor basis. In some embodiments, the dynamic spatial filter component 208 can selectively transmit data from the sensor comprising the plurality of subdivisions of a sensor based in part on at least one of the relevance of the sensor to the learning task and the noise or channel corruption of the sensor.

In some embodiments, the channels can include an array of sensors and the computing device can perform measurements for the dataset using the array of sensors of the channels. In some embodiments, the array of sensors include uniform sensor types or disparate sensor types. In some embodiments, multiple types and levels of dynamic spatial filter components 208 can be implemented. For example, a first dynamic spatial filter component 208 can be configured to receive subdivisions of a sensor in the system to denoise that sensor individually. A second dynamic spatial filter component 208 can be implemented wherein each of the channels of its plurality of channels can receive data from one of a plurality of sensors individually denoised by one of a plurality of first dynamic spatial filter component. In such an embodiment, the first dynamic spatial filters can each reweigh the sensor data and the second dynamic spatial filter component can further reweigh data from multiple sensors. In some embodiments, dynamic spatial filter components, each filtering a sensor by reweighing subdivisions of that sensor, can selectively activate or deactivate their sensor based on, for example, signal corruption or noise or relevance to a learning task. In some embodiments, a dynamic spatial filter component that filters multiple sensors can selectively activate and deactivate sensors based on, for example, signal corruption or noise or relevance to a learning task. Further, dynamic spatial filter components 208 could be implemented through the system at different levels of data processing.

In some embodiments, the dynamic spatial filter includes a weight matrix. In some embodiments, the dynamic spatial filter includes a bias vector. The weight matrix or bias vector can be convenient outputs of the neural network to subsequently apply to the plurality of channels to reweigh them. These forms of data can also provide opportunities to inspect the attention given by the dynamic spatial filter produced to each channel (e.g., by determining a channel contribution metric).

In some embodiments, computing device 204 further has a display. In some embodiments, the dynamic spatial filter can be visualized. For example, the computing device 204 can generate an interface for the display, or transmit data to the interface. The interface can have visual elements or representations for the dynamic spatial filter (e.g., a channel contribution metric). In some embodiments, the dynamic spatial filter can be visualized at the interface in real-time. In some embodiments, the interface can visualize the dynamic spatial filter in real-time using visual elements indicating a relative significance of at least one channel of the plurality of channels using one or more visual elements of the display. In some embodiments, the interface can visualize the dynamic spatial filter in real-time using visual elements indicating signal quality feedback based in part on the learning task using one or more visual elements of the display. For example, some channels may be more critical in performing a trained learning task, and, in that situation, it may be advantageous to ensure that the critical channels register good signal quality even at the possible detriment of signals arising from less critical channels. In some embodiments, the computing device 204 can generate the visual elements for the interface of the display.

In some embodiments, computing device 204 can be further configured to identify an optimal location for hardware corresponding to at least one channel of plurality of channels 202 based in part on the dynamic spatial filter. For example, some applications may prefer certain sensor configurations that target specific signal profiles (e.g., signal profiles of a specific brain structure), and the system can be configured to use the dynamic spatial filter extracted by a neural network to identify that location in a specific context (e.g., with a new user). In some embodiments, the optimal location is determined by the computing device 204 in part by expected signals from an intended target of the at least one channel of the plurality of channels. In some embodiments, the plurality of channels include a plurality of bio-signal sensors, and the dataset aggregates bio-signal data from the bio-signal sensors. The learning task can involve predicting a brain state based in part on the bio-signal data, and the intended target can be a brain structure of a user and the computing device can perform measurements for the bio-signal data using a plurality of bio-signal sensors of the channels. In some embodiments, the plurality of channels include a plurality of microphones, the dataset aggregates audio data from the microphones, and the intended target can be a particular voice in a crowd of individuals or a particular sound in a noisy space. In some embodiments, the expected signals from the intended target are signals expected from a brain structure. In some embodiments, the expected signals can be a general signal profile expected from an intended target.

In some embodiments, the predicting a dynamic spatial filter from the representation of the dataset or the plurality channels using a neural network involves soft-thresholding channels of the plurality channels. In some embodiments, the channel noisiness can be interrogated, and the channel can be weighted to zero (e.g., will not be used to produce the resultant channels (e.g., virtual channels)) where it is found to be above a noisiness threshold.

In some embodiments, computing device 204 is further configured to identify a source space (for example, a source of bio-signals, a source of a voice in a crowded room, a source of a sound in a noisy room) using the dynamic spatial filter. For example, information extracted from the dynamic spatial filter may be used to determine the source of a signal. As a more specific example, the system may be trained and configured to identify where in a body of a user a bio-signal specifically originates. For example, the system can be configured to recover the sources in the brain signal space.

In some embodiments, the system can use the results of the learning task to adjust at least one trainable parameter of at least one of neural network 214 or neural network 216. In such embodiments, it may be possible that the system can engage in further learning in order to adapt to particular use contexts. In some embodiments, the system can fine-tune the dynamic spatial filter each time the system is used. The system can be trained per-device, so that each specific hardware unit on which the system is implemented works optimally given the empirical level of noise on the hardware unit. In some embodiments, trainable parameters can be updated or modified by the computing device 204 based in part on a user profile, environmental conditions (e.g., environmental noise arising from the location), or device usage patterns (e.g., how old or worn the hardware unit is).

In some embodiments, applying the dynamic spatial filter involves adjusting the channels to a form acceptable by the second neural network in the performing a learning task. In some embodiments, the second neural network is trained with input having a specific structure. In some embodiments, a dynamic spatial filter component can reweigh the plurality of channels such that their structure matches a specific structure required by second neural network. This can involve, for example, reweighing the channels such that multiple channels are integrated into one (e.g., dimensionality reduction or channel compression). New ‘channels’ can also be generated if required as well (e.g., dimensionality expansion.

In some embodiments, the system is further configured to selectively transmit at least one channel of the plurality of channels based in part on the dynamic spatial filter. In some embodiments, a dynamic spatial filter component can modify the rate of data sampling from a channel of the plurality of channels based in part on the relevance to a learning task or channel corruption or noise of that channel. In some embodiments, a sensor associated with a channel of the plurality of the channels can be selectively activated or deactivated based in part on the relevance to a learning task or channel corruption or noise of that channel.

In some embodiments, applying the dynamic spatial filter comprises selectively transmitting at least one dynamically reweighed channel. In some embodiments, a dynamic spatial filter component can modify the rate of data transmission from a channel of the plurality of channels based in part on the relevance to a learning task or channel corruption or noise of that channel. In some embodiments, a reweighed channel can be selectively transmitted from a dynamic spatial filter component based in part on the relevance to a learning task or channel corruption or noise of that channel.

In some embodiments, the learning task involves predicting a sleep stage. In some embodiments, the learning task involves detecting pathologies. In some embodiments, the plurality of channels includes a plurality of bio-signal sensors, and the dataset aggregates bio-signal data from the bio-signal sensors. The learning task can involve predicting a brain state based in part on the bio-signal data.

In another aspect, embodiments described herein provide a system for dynamically reweighing a plurality of channels according to relevance given a learning task or channel corruption using a neural network. The system includes a memory, a processor coupled to the memory programmed with executable instructions, and a monitor device. The instructions including an interface for receiving a dataset from a plurality of channels. When executing the instructions the processor extracts a representation of the dataset or the plurality of channels, predicts a dynamic spatial filter from the representation of the dataset or the plurality of channels using a neural network, applies the dynamic spatial filter to dynamically reweigh each of the channels of the plurality channels, and performs a learning task using the reweighed channels and a second neural network. The monitor device includes a plurality of sensors for measuring and collecting the datasets, and a transmitter for transmitting the collected datasets to the interface.

In another aspect, embodiments described herein provide a system for dynamically reweighing a plurality of channels according to relevance given a learning task or channel corruption using a neural network. The system comprising a memory, and a processor coupled to the memory programmed with executable instructions. The instructions including a measuring component for measuring and collecting the datasets using a plurality of sensors for and transmitting the collected datasets to the interface using a transmitter, an interface for receiving a dataset from a plurality of channels; and a reweighing component for extracting a representation of the dataset or the plurality of channels, predicting a dynamic spatial filter from the representation of the dataset or the plurality of channels using a neural network, applying the dynamic spatial filter to dynamically reweigh each of the channels of the plurality channels, and performing a learning task using the reweighed channels and a second neural network.

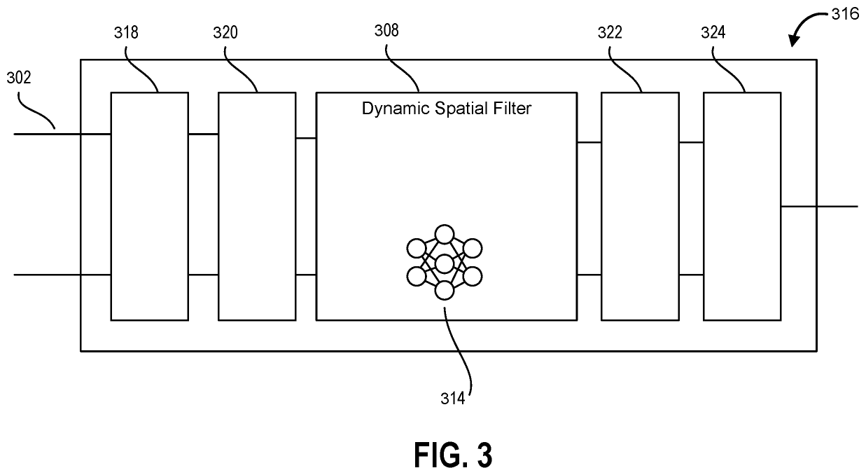

FIG. 3 illustrates a high-level schematic diagram of an implementation of a dynamic spatial filter component within another neural network, according to some embodiments.

FIG. 3 shows a neural network 316 which can receive input from a plurality of channels 302. Neural network 316 comprises layers 318, 320, 322, and 324. Each of the layers 318, 320, 322, and 324 has nodes. The neural network 316 has a dynamic spatial filter component 308.

Dynamic spatial filter component 308 can be another neural network 314. The channels 302 can provide input to a first layer 318 of the neural network 316. In some embodiments layer 318 can have a plurality of layers. A final or last layer 324 of the neural network 316 can also have a plurality of layers. Layer 318 of neural network 316 can process the data received as input according to its learned/trained parameters to generate input to a next layer. The first layer 318 transforms data received as input to generate data for output to the next layer 320 in the neural network 316. Layer 320 can receive the data that is output of layer 318, process the data according to its learned/trained parameters to generate data for output, and provide its output to dynamic spatial filter component 308. In this example, the dynamic spatial filter component 308 can be configured between layers 320 and 322. In other embodiments, the dynamic spatial filter component 308 can be configured between others layers of the neural network 316. Dynamic spatial filter component 308 can predict a dynamic spatial filter from the output of layer 320 using neural network 314 and can apply that dynamic spatial filter to the data that is output of layer 320 to generate filtered data to be received as input by layer 322. That is, the dynamic spatial filter can transform the data output by a layer 320 of the neural network to generate filtered data for input to the next or subsequent layer 322 of the neural network 316. Dynamic spatial filter component 308 can reweigh the plurality of channels 302 while it is being processed by neural network 316 to generate the filtered data.

In some embodiments, the dynamic reweighing can reweigh the channels according to their relevance to a learning task or channel corruption. In some embodiments, this reweighing can reweigh properties of measured data (e.g., frequency ranges) provided by the plurality of channels.

In some embodiments, applying the dynamic spatial filter involves applying the dynamic spatial filter to input of a first layer of the second neural network. In some embodiments, the dynamic spatial filter is applied to a plurality of channels and the second neural network receives the plurality of channels as input.

In some embodiments, applying the dynamic spatial filter involves applying the dynamic spatial filter to output of layer 320 of the neural network 316.

In some embodiments, the dataset involves data that is output of layer 320 of the neural network 316 and the performing a learning task using the reweighed channels and the neural network 316 involves providing the reweighed channels to at least one subsequent layer 322 to the layer of neural network 316.

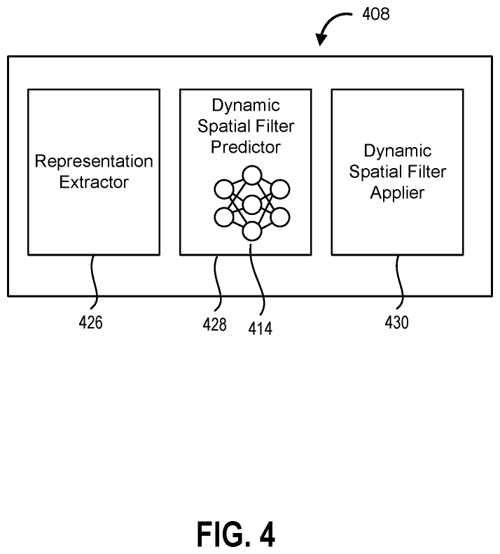

FIG. 4 illustrates a high-level schematic diagram of a dynamic spatial filter component, according to some embodiments.

Dynamic spatial filter component 408 can involve a representation extractor 426, a dynamic spatial filter extractor 428, and a dynamic spatial filter applier 430. Dynamic spatial filter extractor can involves a neural network 414. Dynamic spatial filter component 408 can receive a dataset from a plurality of channels and extract a representation from a dataset or the plurality of channels using representation extractor 426. Dynamic spatial filter component 408 can predict a dynamic spatial filter from the representation extracted from the dataset or the plurality of channels using neural network 414. Dynamic spatial filter component 408 can apply the dynamic spatial filter to reweight the plurality of channels using dynamic spatial filter applier 430.

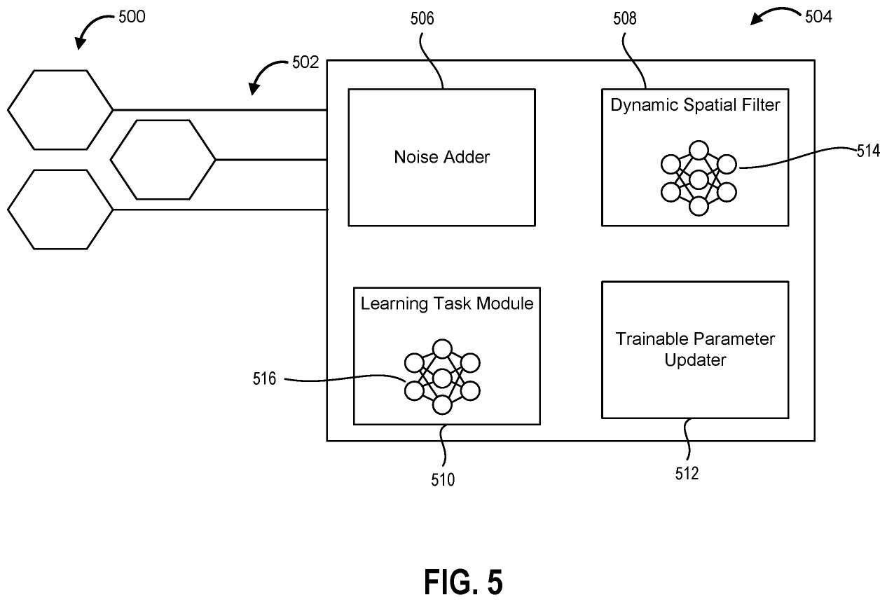

Dynamic spatial filter component 408 can be dynamic spatial filter component 208, 308, or 508 in FIG. 2, FIG. 3, and FIG. 5 respectively. Dynamic spatial filter component 408 can process a dataset prior to the dataset's processing by a second neural network. In some embodiments, dynamic spatial filter component 408 can be integrated between the layers of a second neural network trained to perform a learning task.

FIG. 5 illustrates a high-level schematic diagram of an example learning system for a dynamic spatial filter, according to some embodiments.

FIG. 5 shows learning system 504 which receives input from a plurality of channels 502 and comprises a dynamic spatial filter component 508, a learning task module 510, and a trainable parameter updater 512. Dynamic spatial filter component 508 can comprise a neural network 514. Learning task module 510 can comprise a neural network 516. Optionally, the plurality of channels 502 can receive their input from plurality of sensors 500. Optionally, learning system 504 can further comprise noise adder 506.

In some embodiments, dynamic spatial filter component 508 can be trained using learning system 504. Plurality of sensors 500 can provide data to plurality of channels 502. In some embodiments, noise adder 506 can optionally add noise or channel corruption to the dataset in plurality of channels 502. Dynamic spatial filter component 508 can predict a dynamic spatial filter from the dataset or plurality of channels 502 using neural network 514 and apply that dynamic spatial filter to reweigh the channels. Learning task module 510 can use the reweighed channels to perform a learning task using neural network 516. Trainable parameter updater 512 can then update at least one trainable parameter of at least one of neural network 514 and neural network 516 based on a learning objective.

Once training is complete, the system can be reconfigured, for example in a manner similar to FIG. 2, to perform learning tasks in an application. Furthermore, the functionality and limitations described for the system in FIG. 2 apply equally to the system in FIG. 5 unless otherwise suggested.

For simplicity only one learning system 504 is shown but the system may include multiple computing devices and the components found within can be distributed amongst those computing devices. The computing device components may be connected in various ways including directly coupled, indirectly coupled via a network, and distributed over a wide geographic area and connected via a network (which may be referred to as “cloud computing”).

For example, and without limitation, the computing device may be a server, network appliance, set-top box, embedded device, computer expansion module, personal computer, laptop, personal data assistant, cellular telephone, smartphone device, UMPC tablets, video display terminal, gaming console, electronic reading device, and wireless hypermedia device or any other computing device capable of being configured to carry out the methods described herein.

FIG. 5 is one exemplary embodiment of a system to train dynamic spatial filter component 508. Noise adder 506 may be absent of inactive. Dynamic spatial filter component 508 can be provided between layers of neural network 516 in a manner similar to the system illustrated in FIG. 4. Plurality of channels 502 may provide a dataset stored in a memory rather than a dataset arising from optional plurality of sensors 500.

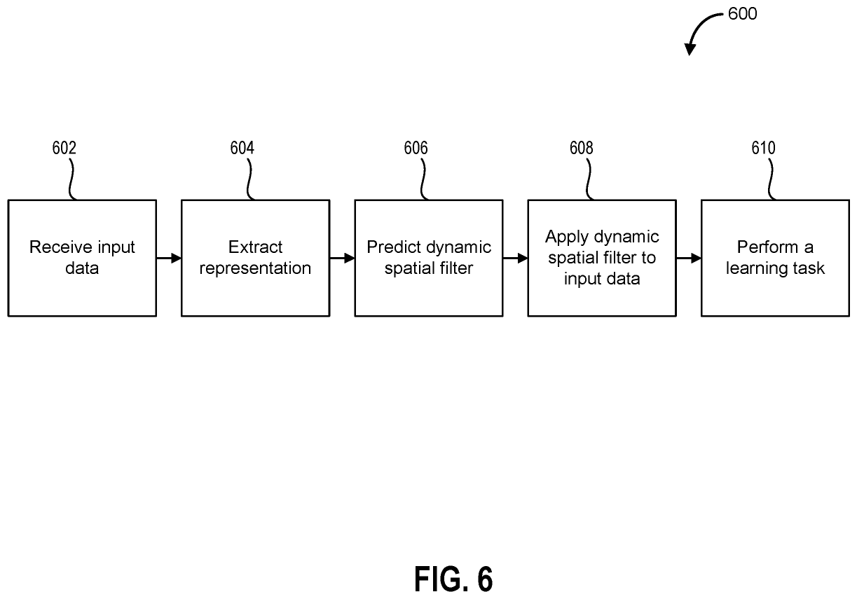

FIG. 6 illustrates a flowchart example of operations using dynamic spatial filtering methods (600), according to some embodiments.

In one aspect, embodiments described herein provide a method of using a neural network to dynamically reweigh a plurality of channels according to relevance given a learning task or channel corruption (600). The method involves receiving a dataset from a plurality of channels (602), each channel of the plurality of channels having data. The method involves extracting a representation of the dataset or the plurality of channels (604), predicting a dynamic spatial filter from the representation of the dataset or the plurality channels using a neural network (606), applying the dynamic spatial filter to dynamically reweigh each of the channels of the plurality channels (608), and performing a learning task using the reweighed channels and a second neural network (610). In some embodiments, the representation is the result of one or more transformations that makes the dataset easier for the neural network to work with. In some embodiments, the method (600) can further involve performing measurements for the dataset using a plurality of sensors. In such embodiments, the each channel may receive the data from a corresponding sensor or the plurality of sensors.

In some embodiments, the dynamic spatial filter comprises unbounded weights to preserve the conceptual connection between channel recombination and spatial filtering. In some embodiments, the dynamic spatial filter predicted by the system is not limited to a linear combination of channel weights. In some embodiments, the weights can be any real number as predicted by the system such that the predicted spatial filters more closely reflect which channels will contribute the most to the system performance.

In some embodiments, neural network is trained for a predictive task. In some embodiments neural network is trained to predict a result based on inputs. Predictive tasks can include tasks such as classification, regression, segmentation, and clustering.

In some embodiments, neural network is trained to perform a related learning task. In some embodiments, neural network is trained to learn new feature spaces. In some embodiments, neural network is trained to create embeddings. Learning tasks can include tasks such as reinforcement learning, density estimation, reconstruction, and generative modelling.

In some embodiments, neural network is used to perform a learning task by performing a predictive task or a related learning task. In such embodiments, the learning task may be completed by another decision-making entity (e.g., another neural network, a machine learning model, a rule-based algorithm, or a person) using the predictive task or the related learning task completed by neural network.

In some embodiments, the representation extracted from the dataset or plurality of channels comprises a first, second, third, or fourth order representation. In some embodiments, the representation comprises spatial covariance, correlational information, or cosine similarity. In some embodiments, the representation captures dependencies between the channels. In some embodiments, the representation comprises a second order representation that comprises at least one of spatial covariance information, correlational information, and cosine similarity to capture dependencies between the plurality of channels. In some embodiments, a second order spatial covariance matrix can be extracted from the dataset or the plurality of channels and vectorized. Such representations can be passed into a neural network to predict a dynamic spatial filter, the neural network having been trained to do so to complete a learning task.

In some embodiments, the method (600) involves applying the dynamic spatial filter (608) by applying the dynamic spatial filter to input of a first layer of the second neural network. In some embodiments, the dynamic spatial filter is applied to a plurality of channels and the second neural network receives the plurality of channels as input.

In some embodiments, the method (600) involves applying the dynamic spatial filter (608) by applying the dynamic spatial filter to output of layer of the second neural network. In some embodiments, the neural network of the dynamic spatial filter is provided between the layers of the second neural network. In such embodiments, the dynamic spatial filter is applied to the output of one layer of the second neural network before that output is provided as input to the next or subsequent layer of the second neural network.

In some embodiments, the representation is at least one of non-linear relational data between the channels such as fractal representations, mutual information, and Granger causality.

In some embodiments, the channels have a plurality of sensors and the method involves performing measurements for the dataset using the plurality of sensors of the channels. The sensors or electrodes can be placed at different positions on the user to capture bio-signal data of the user, for example. In some embodiments, each sensor provides data to a corresponding channel in the plurality of channels. Receiving data (602) is received from the sensors from the plurality of channels. The sensors can capture bio-signal data, such as brainwave or brain activity data, for example. The channels can have different types of sensors. Example sensors include at least one of bio-signal, EEG, EMG, fNIRS, audio-signal, seismographic, radar, telescopic, motion sensors, chemical sensor data, protein sensor data, and video-signal. The data can involve different types of data, such as bio-signal, EEG, EMG, fNIRS, audio-signal, seismographic, radar, telescopic, motion data, chemical sensor data, protein sensor data, and video-signal, for example.

In some embodiments, channels receive data from a plurality of subdivisions of a sensor. Subdivisions can include spatial subdivisions of the sensor, frequency subdivisions of a measured signal, or other divisions that can be used to assess the signal from the sensor. In such embodiments, the system can be configured to determine, for example, a second-order covariance matrix of the subdivisions and, using this, extract and apply a dynamic spatial filter, for example, to denoise the signal by reducing the weight of noisy regions of a signal based on the rest of the signal. In some embodiments, dynamic spatial filter component can reweigh the plurality of channels to reduce noise or channel corruption in the sensor having the plurality of subdivisions of a sensor. In some embodiments, a dynamic spatial filter component can apply a dynamic spatial filter to a plurality of subdivisions of a sensor for each sensor in a system which can denoise the sensors on a sensor-by-sensor basis. In some embodiments, the dynamic spatial filter component can selectively transmit data from the sensor comprising the plurality of subdivisions of a sensor based in part on at least one of the relevance of the sensor to the learning task and the noise or channel corruption of the sensor.

In some embodiments, channels can include an array of sensors and the method includes performing measurements for the dataset using the array of sensors of the channels. In some embodiments, the array of sensors include uniform sensor types or disparate sensor types. In some embodiments, multiple types and levels of dynamic spatial filter components can be implemented. For example, a first dynamic spatial filter component can be configured to receive subdivisions of a sensor in the system to denoise that sensor individually (602). A second dynamic spatial filter component can be implemented wherein each of the channels of its plurality of channels can receive data from one of a plurality of sensors individually denoised by one of a plurality of first dynamic spatial filter component. In such an embodiment, the first dynamic spatial filters can each reweigh the sensor data and the second dynamic spatial filter component can further reweigh data from multiple sensors. In some embodiments, dynamic spatial filter components, each filtering a sensor by reweighing subdivisions of that sensor, can selectively activate or deactivate their sensor based on, for example, signal corruption or noise or relevance to a learning task. In some embodiments, a dynamic spatial filter component that filters multiple sensors can selectively activate and deactivate sensors based on, for example, signal corruption or noise or relevance to a learning task. Further, dynamic spatial filter components could be implemented through the system at different levels of data processing.

In some embodiments, the dynamic spatial filter includes a weight matrix. In some embodiments, the dynamic spatial filter includes a bias vector. The weight matrix or bias vector can be convenient outputs of the neural network to subsequently apply to the plurality of channels to reweigh them. These forms of data can also provide opportunities to inspect the attention given by the dynamic spatial filter produced to each channel (e.g., by determining a channel contribution metric).

In some embodiments, the dataset is made of output of a layer of the second neural network and the method involves performing a learning task using the reweighed channels and the second neural network (610) involves providing the reweighed channels to at least one subsequent layer of the second neural network.

In some embodiments, the method further involves visualizing the dynamic spatial filter. In some embodiments, the method (600) can also involve visualizing the dynamic spatial filter in real-time at an interface. In some embodiments the interface may be an electronic user interface. In some embodiments, visualizing the dynamic spatial filter in real-time can include indicating a relative significance of at least one channel of the plurality of channels using one or more visual elements of the interface. The dynamic special filter extracted by the neural network (see step 606) can be interrogated to determine certain characteristics of the dynamic spatial filter such as channel contribution. The dynamic spatial filter can provide information about how it uses the channels for a learning task (e.g., by inspecting a weight matrix). Such information may be capable of providing a channel's relevance to the learning task or a channel's noise. In some embodiments, the method (600) involves visualizing the dynamic spatial filter in real-time by indicating signal quality feedback based in part on the learning task using one or more visual elements of the interface.

In some embodiments, the method (600) further involves identifying an optimal location for hardware corresponding to at least one channel of the plurality of channels based in part on the dynamic spatial filter. In some embodiments, the optimal location is determined in part by expected signals from an intended target of the at least one channel of the plurality of channels. In some embodiments, the plurality of channels include a plurality of bio-signal sensors and the dataset aggregates bio-signal data from the bio-signal sensors. The learning task can involve predicting a brain state based in part on the bio-signal data, and the intended target can be a brain structure of a user and the method (600) involves performing measurements for the bio-signal data using a plurality of bio-signal sensors of the channels. In some embodiments, the plurality of channels include a plurality of microphones, the dataset aggregates audio data from the microphones, and the intended target can be a particular voice in a crowd of individuals or a particular sound in a noisy space. In some embodiments, the expected signals from the intended target are signals expected from a brain structure. In some embodiments, the expected signals can be a general signal profile expected from an intended target.

In some embodiments, the predicting a dynamic spatial filter from the representation of the dataset or the plurality channels using a neural network (606) involves soft-thresholding channels of the plurality channels. In some embodiments, the channel noisiness can be interrogated, and the channel can be weighted to zero (e.g., will not be used to produce the resultant channels (e.g., virtual channels)) where it is found to be above a noisiness threshold.

In some embodiments, the method (600) further involves identifying a source space (for example, a source of bio-signals, a source of a voice in a crowded room, or a source of a sound in a noisy space) using the dynamic spatial filter. For example, information extracted from the dynamic spatial filter may be used to determine the source of a signal. As a more specific example, the system may be trained and configured to identify from where in a body of a user a bio-signal is specifically originates. For example, the system can be configured to recover the sources in the brain signal space.

In some embodiments, the method (600) further involves using results of the learning task to adjust at least one trainable parameter of at least one of the neural network and the second neural network.

In some embodiments, the applying the dynamic spatial filter (608) involves adjusting the channels to a form acceptable by the second neural network in the performing a learning task. In some embodiments, the second neural network is trained with input having a specific structure. In some embodiments, a dynamic spatial filter component can reweigh the plurality of channels such that their structure matches a specific structure required by second neural network. This can involve, for example, reweighing the channels such that multiple channels are integrated into one (e.g., dimensionality reduction or channel compression). New ‘channels’ can also be generated if required as well (e.g., dimensionality expansion).

In some embodiments, the method further involves selectively transmitting at least one channel of the plurality of channels based in part on the dynamic spatial filter. In some embodiments, a dynamic spatial filter component can modify the rate of data sampling from a channel of the plurality of channels based in part on the relevance to a learning task or channel corruption or noise of that channel. In some embodiments, a sensor associated with a channel of the plurality of the channels can be selectively activated or deactivated based in part on the relevance to a learning task or channel corruption or noise of that channel.

In some embodiments, the applying the dynamic spatial filter (608) involves selectively transmitting at least one dynamically reweighed channel. In some embodiments, a dynamic spatial filter component can modify the rate of data transmission from a channel of the plurality of channels based in part on the relevance to a learning task or channel corruption or noise of that channel. In some embodiments, a reweighed channel can be selectively transmitted from a dynamic spatial filter component based in part on the relevance to a learning task or channel corruption or noise of that channel.

In some embodiments, the learning task involves predicting a sleep stage. In some embodiments, the learning task involves detecting pathologies. In some embodiments, the plurality of channels includes a plurality of bio-signal sensors, and the dataset aggregates bio-signal data from the bio-signal sensors. The learning task can involve predicting a brain state based in part on the bio-signal data.

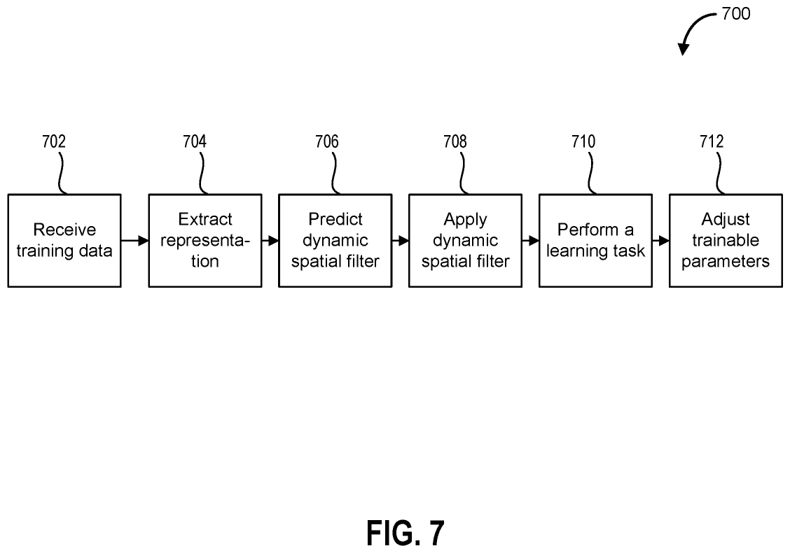

FIG. 7 illustrates a flowchart example of operations of a method of training a dynamic spatial filter component, according to some embodiments.

In one aspect, embodiments described herein provide a method of adjusting trainable parameters of neural networks to dynamically reweigh a plurality of channel according to relevance given a learning task or channel corruption (700). The method involves receiving a dataset from a plurality of channels (702), each channel of the plurality of channels having data. The method involves extracting a representation of the dataset or the plurality of channels (704), predicting a dynamic spatial filter from the representation of the dataset or the plurality of channels using a neural network (706), applying the dynamic spatial filter to dynamically reweigh each of the channels of the plurality of channels (708), performing a learning task using the reweighed channels and a second neural network (710), and using a learning objective to adjust at least one trainable parameter of at least one of the neural network and the second neural network (712). In some embodiments, the representation is the result of one or more transformations that makes the dataset easier for the neural network to work with. In some embodiments, the method (700) can include performing measurements for the dataset using a plurality of sensors. In such embodiments, the each channel may receive the data from a corresponding sensor or the plurality of sensors.

In some embodiments, the dynamic spatial filter comprises unbounded weights to preserve the conceptual connection between channel recombination and spatial filtering. In some embodiments, the dynamic spatial filter predicted by the system is not limited to a linear combination of channel weights. In some embodiments, the weights can be any real number as predicted by the system such that the predicted spatial filters more closely reflect which channels will contribute the most to the system performance.

The learning method illustrated in FIG. 7 can be varied to correspond to any implementation method for a dynamic spatial filter component. The learning method will preferentially imitate the expected implementation configuration. For example, a dynamic spatial filter component that will be provided between two layers of a second neural network (such as the configuration of FIG. 3) in operation, can be trained in a similar configuration.

In some embodiments, the learning objective involves minimizing a difference between predicted results and expected results. In some embodiments, trainable parameters of the neural network or the second neural network are updated to reduce a degree of difference between predicted results and expected results. In some embodiments, trainable parameters of the neural network or the second neural network are updated to increase a degree of similarity between predicted results and expected results.

In some embodiments, the representation extracted from the dataset or plurality of channels comprises a first, second, third, or fourth order representation. In some embodiments, the representation comprises spatial covariance, correlational information, or cosine similarity. In some embodiments, the representation captures dependencies between the channels. In some embodiments, the representation comprises a second order representation that comprises at least one of spatial covariance information, correlational information, and cosine similarity to capture dependencies between the plurality of channels. In some embodiments, a second order spatial covariance matrix can be extracted from the dataset or the plurality of channels and vectorized.

Such representations can be passed into a neural network to predict a dynamic spatial filter, the neural network having been trained to do so to complete a learning task.

In some embodiments, the method (700) involves applying the dynamic spatial filter (708) by applying the dynamic spatial filter to input of a first layer of the second neural network. In some embodiments, the dynamic spatial filter is applied to a plurality of channels and the second neural network receives the plurality of channels as input.

In some embodiments, the method (700) involves applying the dynamic spatial filter (708) by applying the dynamic spatial filter to output of layer of the second neural network. In some embodiments, the neural network of the dynamic spatial filter is provided between the layers of the second neural network. In such embodiments, the dynamic spatial filter is applied to the output of one layer of the second neural network before that output is provided as input to the next or subsequent layer of the second neural network.

In some embodiments, the representation is at least one of non-linear relational data between the channels such as fractal representations, mutual information, and Granger causality.

In some embodiments, the channels have a plurality of sensors and the method involves performing measurements for the dataset using the plurality of sensors of the channels. The sensors or electrodes can be placed at different positions on the user to capture bio-signal data of the user, for example. In some embodiments, each sensor provides data to a corresponding channel in the plurality of channels. Receiving data (702) is received from the sensors from the plurality of channels. The sensors can capture bio-signal data, such as brainwave or brain activity data, for example. The channels can have different types of sensors. Example sensors include at least one of bio-signal, EEG, EMG, fNIRS, audio-signal, seismographic, radar, telescopic, motion sensors, chemical sensor data, protein sensor data, and video-signal. The data can involve different types of data, such as bio-signal, EEG, EMG, fNIRS, audio-signal, seismographic, radar, telescopic, motion data, chemical sensor data, protein sensor data, and video-signal, for example.

In some embodiments, channels receive data from a plurality of subdivisions of a sensor.

Subdivisions can include spatial subdivisions of the sensor, frequency subdivisions of a measured signal, or other divisions that can be used to assess the signal from the sensor. In such embodiments, the system can be configured to determine, for example, a second-order covariance matrix of the subdivisions and, using this, extract and apply a dynamic spatial filter, for example, to denoise the signal by reducing the weight of noisy regions of a signal based on the rest of the signal. In some embodiments, dynamic spatial filter component can reweigh the plurality of channels to reduce noise or channel corruption in the sensor having the plurality of subdivisions of a sensor. In some embodiments, a dynamic spatial filter component can apply a dynamic spatial filter to a plurality of subdivisions of a sensor for each sensor in a system which can denoise the sensors on a sensor-by-sensor basis. In some embodiments, the dynamic spatial filter component can selectively transmit data from the sensor comprising the plurality of subdivisions of a sensor based in part on at least one of the relevance of the sensor to the learning task and the noise or channel corruption of the sensor.

In some embodiments, channels can include an array of sensors and the method includes performing measurements for the dataset using the array of sensors of the channels. In some embodiments, the array of sensors include uniform sensor types or disparate sensor types. In some embodiments, multiple types and levels of dynamic spatial filter components can be implemented. For example, a first dynamic spatial filter component can be configured to receive subdivisions of a sensor in the system to denoise that sensor individually (702). A second dynamic spatial filter component can be implemented wherein each of the channels of its plurality of channels can receive data from one of a plurality of sensors individually denoised by one of a plurality of first dynamic spatial filter component. In such an embodiment, the first dynamic spatial filters can each reweigh the sensor data and the second dynamic spatial filter component can further reweigh data from multiple sensors. In some embodiments, dynamic spatial filter components, each filtering a sensor by reweighing subdivisions of that sensor, can selectively activate or deactivate their sensor based on, for example, signal corruption or noise or relevance to a learning task. In some embodiments, a dynamic spatial filter component that filters multiple sensors can selectively activate and deactivate sensors based on, for example, signal corruption or noise or relevance to a learning task. Further, dynamic spatial filter components could be implemented through the system at different levels of data processing.

In some embodiments, the dynamic spatial filter includes a weight matrix. In some embodiments, the dynamic spatial filter includes a bias vector. The weight matrix or bias vector can be convenient outputs of the neural network to subsequently apply to the plurality of channels to reweigh them. These forms of data can also provide opportunities to inspect the attention given by the dynamic spatial filter produced to each channel (e.g., by determining a channel contribution metric).

In some embodiments, the dataset is made of output of a layer of the second neural network and the method involves performing a learning task using the reweighed channels and the second neural network (710) involves providing the reweighed channels to at least one subsequent layer of the second neural network.

In some embodiments, the method (700) further involves visualizing the dynamic spatial filter. In some embodiments, the method (700) can also involve visualizing the dynamic spatial filter in real-time at an interface. In some embodiments the interface may be an electronic user interface. In some embodiments, visualizing the dynamic spatial filter in real-time can include indicating a relative significance of at least one channel of the plurality of channels using one or more visual elements of the interface. The dynamic special filter extracted by the neural network (see step 606) can be interrogated to determine certain characteristics of the dynamic spatial filter such as channel contribution. The dynamic spatial filter can provide information about how it uses the channels for a learning task (e.g., by inspecting a weight matrix). Such information may be capable of providing a channel's relevance to the learning task or a channel's noise. In some embodiments, the method (700) involves visualizing the dynamic spatial filter in real-time by indicating signal quality feedback based in part on the learning task using one or more visual elements of the interface.

In some embodiments, the method (700) further involves identifying an optimal location for hardware corresponding to at least one channel of the plurality of channels based in part on the dynamic spatial filter. In some embodiments, the optimal location is determined in part by expected signals from an intended target of the at least one channel of the plurality of channels. In some embodiments, the plurality of channels include a plurality of bio-signal sensors and the dataset aggregates bio-signal data from the bio-signal sensors. The learning task can involve predicting a brain state based in part on the bio-signal data, and the intended target can be a brain structure of a user the method (700) involves performing measurements for the bio-signal data using a plurality of bio-signal sensors of the channels. In some embodiments, the plurality of channels include a plurality of microphones, the dataset aggregates audio data from the microphones, and the intended target can be a particular voice in a crowd of individuals or a particular sound in a noisy space. In some embodiments, the expected signals from the intended target are signals expected from a brain structure. In some embodiments, the expected signals can be a general signal profile expected from an intended target.

In some embodiments, the predicting a dynamic spatial filter from the representation of the dataset or the plurality channels using a neural network (706) involves soft-thresholding channels of the plurality channels. In some embodiments, the channel noisiness can be interrogated, and the channel can be weighted to zero (e.g., will not be used to produce the resultant channels (e.g., virtual channels)) where it is found to be above a noisiness threshold.

In some embodiments, the method (700) further involves identifying a source space (for example, a source of bio-signals, a source of a voice in a crowded room, or a source of a sound in a noisy space) using the dynamic spatial filter. For example, information extracted from the dynamic spatial filter may be used to determine the source of a signal. As a more specific example, the system may be trained and configured to identify from where in a body of a user a bio-signal is specifically originates. For example, the system can be configured to recover the sources in the brain signal space.

In some embodiments, the applying the dynamic spatial filter (708) involves adjusting the channels to a form acceptable by the second neural network in the performing a learning task. In some embodiments, the second neural network is trained with input having a specific structure. In some embodiments, a dynamic spatial filter component can reweigh the plurality of channels such that their structure matches a specific structure required by second neural network. This can involve, for example, reweighing the channels such that multiple channels are integrated into one (e.g., dimensionality reduction or channel compression). New ‘channels’ can also be generated if required as well (e.g., dimensionality expansion).

In some embodiments, the method (700) further involves selectively transmitting at least one channel of the plurality of channels based in part on the dynamic spatial filter. In some embodiments, a dynamic spatial filter component can modify the rate of data sampling from a channel of the plurality of channels based in part on the relevance to a learning task or channel corruption or noise of that channel. In some embodiments, a sensor associated with a channel of the plurality of the channels can be selectively activated or deactivated based in part on the relevance to a learning task or channel corruption or noise of that channel.

In some embodiments, the applying the dynamic spatial filter (708) involves selectively transmitting at least one dynamically reweighed channel. In some embodiments, a dynamic spatial filter component can modify the rate of data transmission from a channel of the plurality of channels based in part on the relevance to a learning task or channel corruption or noise of that channel. In some embodiments, a reweighed channel can be selectively transmitted from a dynamic spatial filter component based in part on the relevance to a learning task or channel corruption or noise of that channel.

In some embodiments, the learning task involves predicting a sleep stage. In some embodiments, the learning task involves detecting pathologies. In some embodiments, the plurality of channels includes a plurality of bio-signal sensors, and the dataset aggregates bio-signal data from the bio-signal sensors. The learning task can involve predicting a brain state based in part on the bio-signal data.

In some embodiments, the method (700) can further involves adding noise or channel corruption to the dataset or the plurality of channels prior to the extracting a representation of the dataset or the plurality of channels (704). In some embodiments, the noise or channel corruption can include at least one of additive white noise, spatially uncorrelated additive white noise, pink noise, simulated structured noise, and real noise. Training the neural network can involve generating noise in the dataset to simulate noise conditions (e.g., conditions similar to the ultimate context in which the neural network will operate).

In some embodiments, the plurality of channels can provide a dataset that has been stored from previous applications. In some embodiments, the plurality of channels can provide a dataset generated to simulate training applications.

In some embodiments, the devices, systems, and methods described herein can be used to assist in obtaining clinically relevant data, determining the cognitive skills of a user, determining if instruction is effective, or using bio-markers to, for example, adapt treatments. The system can be used to, for example, determine a biological state of a user, based on a plurality of bio-signal sensors. This determination can be framed as the end learning task for the system or method (e.g., the system or method is configured to predict whether or not a biological state exists) or it can framed as a learning task to assist the end goal of the system (e.g., the system predicts the presence or absence of a biological state and the system uses this prediction to further determine another diagnosis).

### Experimental Results: Robust Learning From Corrupted EEG With Dynamic Spatial Filtering

The following describes a non-limiting, experimental embodiment of the systems, methods, devices described herein directed specifically to the dynamic spatial filtering of EEG devices.

### Computational Considerations

When working with deep neural networks, various training hyperparameters must be set, including the type of optimizer, learning rate schedule, batch size, regularization strength (number of training epochs, weight decay, dropout) and parameter initialization scheme. In all experiments, the AdamW optimizer was used with β₁=0:9, β₂=0.999, a learning rate of 10⁻³ and cosine annealing. The parameters of all neural networks were randomly initialized using uniform He initialization. Dropout was applied to f_{θ}'s fully connected layers at a rate of 50% and weight decay was applied to the trainable parameters of all layers of both f_{θ} and h_{θ}_{_{DSF}}. Moreover, during training, the loss was weighted to help counter class imbalance. Some hyperparameters were tuned on a dataset-specific basis and are described along with the datasets.

Deep learning and baseline models were trained using a combination of the braindecode, MNE-Python, PyTorch, pyRiemann, mne-features and scikit-learn packages. Finally, deep learning models were trained on 1 or 2 Nvidia Tesla V100 or P4 GPUs for anywhere from a few minutes to 7 h, depending on the amount of data, early stopping and GPU configuration.

### Downstream Tasks

Noise robustness was studied through two EEG classification downstream tasks: pathology detection and sleep staging. First, sleep staging, a critical step in sleep monitoring, can allow the diagnosis and study of sleep disorders such as apnea and narcolepsy. This 5-class classification problem consists in predicting which sleep stage (W (wake), N1, N2, N3 (different levels of sleep) and R (rapid eye movement periods)) an individual is in in non-overlapping 30-s windows of overnight recordings. Sleep staging is usually carried out on data collected in sleep clinics where both the environment and instrumentation are controlled by experts. This procedure, called polysomnography (PSG), was recently translated to at-home settings thanks to the availability of mobile EEG devices. Importantly, this now allows monitoring an individual in his usual sleep environment, which is difficult in the clinic. The handling of corrupted channels in overnight recordings has not been addressed in a comprehensive manner as channel corruptions is less likely to occur in clinical and laboratory settings than in real-world settings.

Second, the pathology detection task aims at detecting neurological conditions such as epilepsy and dementia from an individual's EEG. In a simplified formulation this gives rise to a binary classification problem where recordings have to be classified as either pathological or non-pathological. Such recordings are typically carried out in well-controlled settings (e.g., in a hospital) where sources of noise can be monitored and mitigated in real-time by experts. To test automated pathology detection performance in the context of mobile EEG acquisition, a limited set of electrodes was used. This more closely simulates at-home neurological screening with mobile EEG devices (which can help reach more patients, e.g., in geographically remote regions or with poor access to neurology expertise).

### Compared Methods

The performance of the proposed DSF and data augmentation method was compared to other approaches. In total, combinations of three machine learning pipelines were contrasted with three different noise handling strategies.

The following machine learning pipelines are considered: (1) end-to-end deep learning (with and without the DSF module) from raw signals, (2) filter-bank covariance matrices with Riemannian tangent space projection and logistic regression (herein after “Riemann”), and (3) handcrafted features and random forest (RF).

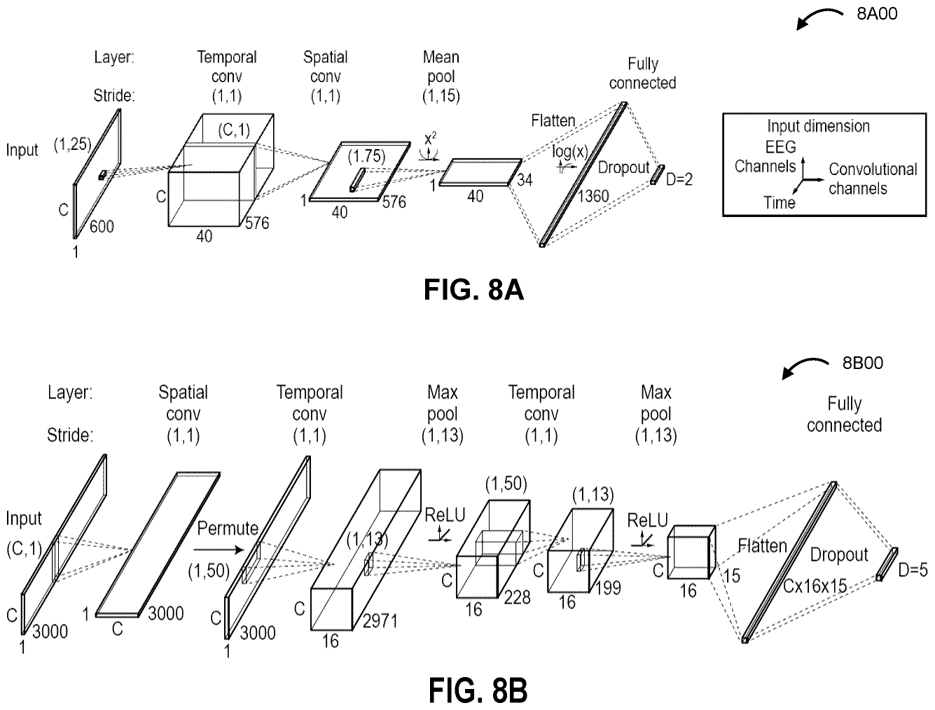

ConvNet architectures were used as f_{θ} in deep learning pipelines. The ConvNets f_{θ} used in the experiments are illustrated in FIG. 8A and FIG. 8B.

FIG. 8A illustrates neural network architectures f_{θ} (8A00) used in pathology detection, according to some embodiments.

FIG. 8B illustrates neural network architectures f_{θ} (8B00) used in sleep staging experiments, according to some embodiments.

For pathology detection, the ShallowNet architecture was used which parametrizes the frequency-band common spatial patterns (FBCSP) pipeline. It was used without modifying the architecture, yielding a total of 13,482 trainable parameters when C=6. For sleep staging, a 3-layer ConvNet was used which takes 30-s windows as input, with a total of 18,457 trainable parameters when C=4 and an input sampling frequency of 100 Hz. Finally, when evaluating DSF, modules m_{DSF} were added before the input layer of each neural network. The input dimensionality of m_{DSF} depends on the chosen spatial information extraction transform Φ(X): it was either C (log-variance) or C(C+1)/2 (vectorized covariance matrix). The hidden layer size of m_{DSF} was fixed to C² units, while the output layer size depends on the chosen C′. The DSF modules added between 420 and 2,864 trainable parameters to those of f_{θ} depending on the configuration.

The Riemann pipeline first applied a filter bank to the input EEG, yielding narrow-band signals in the 7 bands bounded by (0.1, 1.5, 4, 8, 15, 26, 35, 49) Hz. Next, covariance matrices were estimated per window and frequency band using the OAS algorithm. Covariance matrices were then projected into their Riemannian tangent space using the Wasserstein distance to estimate the reference point. The vectorized covariance matrices with dimensionality of C(C+1)/2 were finally z-score normalized using the mean and standard deviation of the training set, and fed to a linear logistic regression classifier.

The handcrafted features baseline, relied on 21 different feature types: mean, standard deviation, root mean square, kurtosis, skewness, quantiles (10, 25, 75 and 90th), peak-to-peak amplitude, frequency log-power bands between (0, 2, 4, 8, 13, 18, 24, 30, 49) Hz as well as all their possible ratios, spectral entropy, approximate entropy, SVD entropy, Hurst exponent, Hjorth complexity, Hjorth mobility, line length, wavelet coefficient energy, Higuchi fractal dimension, number of zero crossings, SVD Fisher information and phase locking value. This resulted in 63 univariate features per EEG channel, along with

$$( \begin{matrix}C \\ 2\end{matrix} )$$

bivariate features, which were concatenated into a single vector of size

$$63 \times C + ( \begin{matrix}c \\ 2\end{matrix} )$$

(e.g., 395 for C=6). In the event of non-finite values in the feature representation of a window, missing values were inputted feature-wise using the mean of the feature computed over the training set. Finally, feature vectors were fed to a random forest model.

When applying other pipelines to pathology detection experiments, the input representations were aggregated recording-wise as each recording has a single label (i.e., pathological or not). This was done on covariance matrices using the geometric mean and the handcrafted features using the median. Deep learning models, on the other hand, were trained on non-aggregated windows, but their performance was evaluated recording-wise by averaging the predictions over windows within each recording. Hyperparameter selection for logistic regression and random forest models is described.

Table 2 shows selected hyperparameters for experiments on number of channels.

| | | Number of channels | | |
|---|---|---|---|---|
| Model | Hyperparameter | 2 | 6 | 21 |
| Random Forest (RF) | Number of trees | 300 | 300 | 300 |
| | Tree depth | 17 | 21 | 19 |
| | Criterion | entropy | Gini | entropy |
| | Features | all | all | All |
| Logistic regression (LR) | C | 0.1 | 0.1 | 0.001 |

Table 3 shows selected hyperparameters for experiments on denoising strategies.

| | | | Denoising strategy | | |
|---|---|---|---|---|---|
| | | | No | Auto- | Data aug- |
| Dataset | Model | Hyperparameter | denoising | reject | mentation |
| TUAB | RF | Number of trees | 300 | 300 | 300 |
| | | Tree depth | 21 | 13 | 17 |
| | | Criterion | Gini | entropy | entropy |
| | | Features | all | all | All |
| | LR | C | 0.1 | 0.1 | 0.01 |
| PC18 | RF | Number of trees | 300 | 300 | 300 |
| | | Tree depth | 15 | 15 | 17 |
| | | Criterion | entropy | Gini | Entropy |
| | | Features | sqrt | sqrt | sqrt |
| | LR | C | 1 | 1 | 10 |
| ISD | RF | Number of trees | 300 | 300 | 300 |
| | | Tree depth | 9 | 9 | 11 |
| | | Criterion | entropy | entropy | Entropy |
| | | Features | all | sqrt | Sqrt |
| | LR | C | 0.1 | 0.1 | 10⁵ |

A grid-search over hyperparameters of the random forest and logistic regression classifiers was performed with 3-fold cross-validation on combined training and validation sets. This search was performed for each reported experimental configuration, i.e., number of channels, each denoising strategy (no denoising, Autoreject and data augmentation) and each dataset (TUAB, PC18 and ISD).

For random forest (RF) models, the number of trees (n estimators) were first tuned while fixing all other hyperparameters to their default value (https://scikit-learn.org/0.22/modules/generated/sklearn.ensemble.RandomForestClassifier.html). Validation performance peaked below 260 trees on both TUAB (with 21 channels) and PC18 (with 6 channels), and so the number of trees were fixed to 300 for all models. This turned out to be a good trade-off between model performance and computational costs. For each experiment, the depths of trees among {13, 15, 17, 19, 21, 23, 25}, the split criterion between Gini and entropy, and the fraction of selected features used in each tree among ‘sqrt’ (the square-root of the number of features used), ‘log2’ (the logarithm in base 2 of the number of features is used), and using all features were selected by cross-validation. For logistic regression models, the regularization parameter C was chosen among {10⁴, 10⁻³, . . . , 10}. The search was expanded on ISD as performance did not peak in the ranges considered above by adding the following values to the search space: depth {1,3,5,7,9,11} and C in {10², 10³, 10⁴, 10⁵}.

The selected hyperparameter configurations are listed in Tables 2 and 3 for the experiments of Performance of existing methods degrades under channel corruption and Attention and data augmentation mitigates performance loss under channel corruption, respectively. Once the best hyperparameters for an experimental configuration were identified, the training and validation sets were combined into a single set on which the model with the best hyperparameters was finally trained.

The machine learning approaches described above were combined with the following noise handling strategies: (1) no denoising, i.e., models are trained directly on the data without explicit or implicit denoising, (2) Autoreject, an automated correction pipeline, and (3) data augmentation that randomly corrupts channels during training.

Autoreject is a denoising pipeline that explicitly handles noisy epochs and channels in a fully automated manner. First, using a cross-validation procedure, it finds optimal channel-wise peak-to-peak amplitude thresholds to be used to identify bad channels in each window separately. If more than k channels are bad, the epoch is rejected. Otherwise, up to p bad channels are reconstructed using the good channels with spherical splines interpolation. In pathology detection experiments, Autoreject was allowed to reject bad epochs, as classification was performed recording-wise. For sleep staging experiments however, epochs were not rejected as one prediction per spoch was needed, but Autoreject was still used to automatically identify and interpolate bad channels. In both cases, default values were used for all parameters as provided in the Python implementation (https://github.com/autoreject/autoreject), except for the number of cross-validation folds, which was set to 5.

Finally, data augmentation consists of artificially corrupting channels during training to promote invariance to missing channels. When training neural networks, the data augmentation transform was applied on-the-fly to each batch. For feature-based methods, augmented datasets were instead precomputed by applying the augmentation multiple times to each window (10 for pathology detection, 5 for sleep staging), and then extracting features from augmented windows.

### Data

Table 4 shows the description of the datasets used in this study.

| | TUAB | PC18 | ISD |
|---|---|---|---|
| Recording settings | Hospital | Sleep clinic | At-home |
| # recording | 2993 | 994 | 98 |
| # unique subjects | 2329 | 994 | 67 |
| Sampling frequency | 250, 256 or 512 | 200 | 256 |
| (Hz) | | | |
| # EEG channels | 27 to 36 | 6 | 4 |
| Reference | Common average | M1 or M2 | Fpz |
| Labels | Normal, abnormal | W, N1, N2, | W, N1, N2, |
| | | N3, R | N3, R |

Approaches were compared on three datasets: for pathology detection on the TUH Abnormal EEG dataset and for sleep staging on both the Physionet Challenge 2018 dataset and an internal dataset of mobile overnight EEG recordings.

The TUH Abnormal EEG dataset v2.0.0 (TUAB) contains 2,993 recordings of 15 minutes or more from 2,329 different patients who underwent a clinical EEG exam in a hospital setting. Each recording was labeled as “normal” (1,385 recordings) or “abnormal” (998 recordings) based on detailed physician reports. Most recordings were sampled at 250 Hz and comprised between 27 and 36 electrodes. The corpus is already divided into a training and an evaluation set with 2, 130 and 253 recordings each. The mean age across all recordings is 49.3 years old (min: 1, max: 96) and 53.5% of recordings are of female patients. The TUAB data was preprocessed in the following manner. The first minute of each recording was cropped to remove noisy data that occurs at the beginning of recordings. Longer files were cropped such that a maximum of 20 minutes was used from each recording. Then, 21 channels common to all recordings were selected (Fp1, Fp2, F7, F8, F3, Fz, F4, A1, T3, C3, Cz, C4, T4, A2, T5, P3, Pz, P4, T6, O1 and O2). EEG channels were downsampled to 100 Hz and clipped at ±800 μV. Finally, non-overlapping 6-s windows were extracted, yielding windows of size (600×21). Deep learning models were trained on TUAB with a batch size of 256 and weight decay of 0.01.

The Physionet Challenge 2018 (PC18) dataset contains recordings from a total of 1,983 different individuals with (suspected) sleep apnea whose EEG, EOG, chin EMG, respiration air flow and oxygen saturation were monitored overnight. Bipolar EEG channels F3-M2, F4-M1, C3-M2, C4-M1, O1-M2 and O2-M1 were recorded at 200 Hz. Sleep stage annotations were obtained from 7 trained scorers following the AASM manual (W, N1, N2, N3 and R). The analysis focused on a subset of 994 recordings for which these annotations are publicly available. In this subset of the data, mean age is 55 years old (min: 18, max: 93) and 33% of participants are female. For PC18, the EEG was first filtered using a 30 Hz FIR lowpass filter with a Hamming window, to reject higher frequencies that are not critical for sleep staging. The EEG channels were then downsampled by a factor of two to 100 Hz to reduce the dimensionality of the input data. Non-overlapping windows of 30 s of size (3000×2) were finally extracted. Experiments on PC18 used a batch size of 64 and weight decay of 0.001.

The approaches described herein were tested on real-world mobile EEG data, in which channel corruption is likely to occur naturally (the ISD dataset). An internal dataset of overnight sleep recordings collected with mobile EEG devices for at-home use. The mobile EEG device was a four-channel dry EEG device (TP9, Fp1, Fp2, TP10, referenced to Fpz), sampled at 256 Hz. The mobile EEG device may also be used for event-related potentials research, brain performance assessment, research into brain development, sleep staging, and stroke diagnosis, among others. A total of 98 partial and complete overnight recordings (mean duration: 6.3 h) from 67 unique users were selected and annotated by a trained scorer following the AASM manual. Despite the derivations being different from the common montage used in polysomnography, the typical microstructure necessary to identify sleep stages, e.g., sleep spindles, k-complexes and slow waves can be easily seen in all four channels. Therefore sleep stage annotations were obtained from actual EEG activity rather than ocular or muscular artifacts. Mean age across all recordings is 37.9 years old (min: 21, max: 74) and 45.9% of recordings are of female users. Preprocessing of ISD data was the same as for PC18, with the following differences: (1) channels were downsampled to 128 Hz, (2) missing values (occurring when the wireless connection is weak or Bluetooth packets are lost) were replaced by linear interpolation using surrounding valid samples, (3) after filtering and downsampling, the samples which overlapped with the original missing values were replaced by zeros, and (4) channels were zero-meaned window-wise. A batch size of 64 and weight decay of 0.01 was used for ISD experiments.

The Internal Sleep Dataset (ISD) is a collection of at-home overnight recordings. These recordings were purposefully selected to evaluate sleep staging algorithms in challenging mobile EEG conditions and therefore include recordings with highly corrupted channels. Overall, sources of noise are generally more common and noise can be stronger and/or more prevalent in these recordings than in typical sleep datasets collected under controlled laboratory conditions (e.g., PC18).

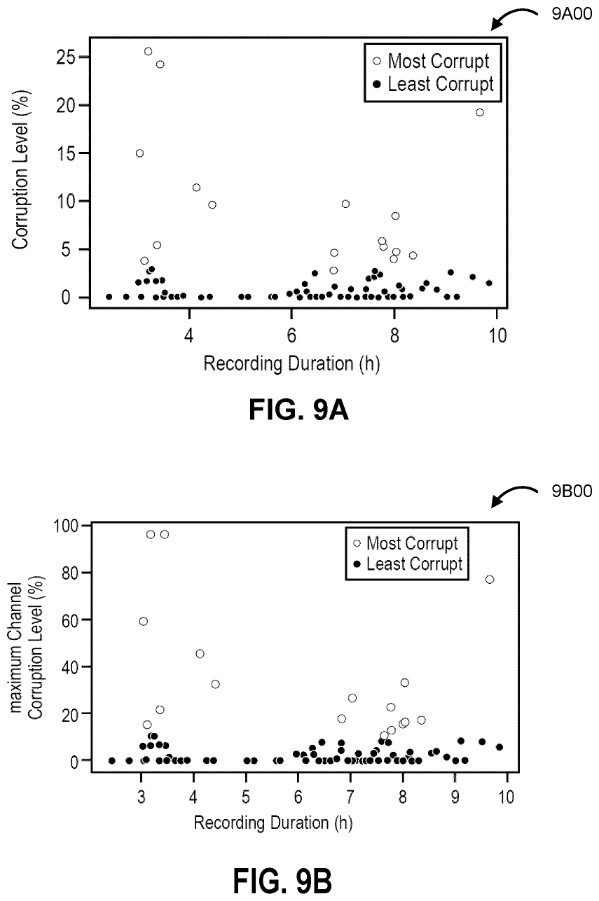

To characterize the prevalence of channel corruption in ISD recordings, the variance and the slope of the power spectral density (PSD) of each EEG channel across 30-s windows was inspected. Variance is a good measure of signal quality (for instance, the DSFd variant received log-variance as input in the experiments), while the spectral slope is indicative of the frequency content of the noise and allows distinguishing between channel corruption (which yields flatter spectra) and artifacts (often displaying strong low frequencies, e.g., eye movements). Simple thresholds set empirically on these two markers allowed approximate detection of channel corruption events. Specifically, a channel in a window was flagged as “corrupted” if its log₁₀-log₁₀ spectral slope between 0.1 and 30 Hz was above −0.5 (unitless) and its variance was above 1,000 μV². A recording-wise channel corruption metric was computed by computing the fraction of channels and windows which were flagged as corrupted. About two-thirds of the recordings had no channel corruption according to this metric, while the remaining had a value of up to 96.4% (FIG. 9B).

FIG. 9A illustrates the corruption percentage of the 98 recordings of ISD (9A00), according to some embodiments. FIG. 9B illustrates the corruption channel percentage of the most corrupted channel of each of the 98 recordings of ISD (9B00), according to some embodiments. Each point represents a single recording. The 17 most corrupted recordings (white) were used as test set in the Attention and data augmentation mitigates performance loss under channel corruption experiments.

In recording with channel corruption, half of the corruption events (defined as continuous block of epochs flagged as corrupted) lasted for 1.5 min or less, suggesting a large portion of the corruption happened intermittently, e.g., due to the temporary displacement of the electrodes relative to the head. Some of the corruption events however lasted much longer, for instance up to 88 min in one case. These longer corruption events are likely due to bad connection between the skin and the electrode or to problems with the instrumentation.

For experiments on ISD, the 81 cleanest recordings (i.e., with the lowest corruption fraction) were selected for training and validation and the 17 noisiest recordings were kept for testing. This procedure allowed testing whether a model trained on clean data with DSF and data augmentation could perform well even when random channel corruption was introduced at inference time.

The available recordings were split from PC18 and TUAB into training, validation and testing sets such that the examples from each recording were only in one of the sets, such that recording used for testing were not used for training or validation. For TUAB, the provided evaluation set was used as the test set. The recordings in the development set were split 80-20% into a training and a validation set. Therefore, 2,171, 543 and 276 recordings were used in the training, validation and testing sets. For PC18, a 60-20-20% random split was used, meaning there were 595, 199 and 199 recordings in the training, validation and testing sets respectively. Finally, for Internal Sleep Dataset (ISD), the 17 most corrupted recordings were retained for the test set and randomly split the remaining 81 recordings into training and validation sets (65 and 16 recordings, respectively). This was done to emulate a situation where training data is mostly clean, and strong channel corruption occurs unexpectedly at test time. Hyperparameter selection was performed on each of the three datasets using a cross-validation strategy on the combined training and validation sets.

Training was repeated on different training-validation splits (two for PC18, three for TUAB and ISD). Neural networks and random forests were trained three times per split on TUAB and ISD (two times on PC18) with different parameter initializations. Training ran for at most 40 epochs or until the validation loss stopped decreasing for a period of a least 7 epochs on TUAB and PC18 (a maximum of 150 epochs and patience of 30 for ISD, given the smaller size of the dataset).

Finally, accuracy was used to evaluate model performance for pathology detection experiments, while balanced accuracy (bal acc), defined as the average per-class recall, was used for sleep staging due to important class imbalance (the N2 class is typically much more frequent than other classes).

### Evaluation Under Noisy Conditions

The impact of noise on downstream performance and on the predicted DSF filters was evaluated in three steps. First, the input EEG windows of TUAB and PC18 was artificially corrupted by using a similar process to the data augmentation strategy (Equation 7). The same values for η, and p were used, but used a single mask v per recording such that the set of corrupted channels remained the same across a recording. Before corrupting, a few EEG channels were subsampled to recreate the sparse montages settings of TUAB (Fp1, Fp2, T3, T4, Fz, Cz) and PC18 (F3-M2, F4-M1, O1-M2, O2-M1). Downstream performance was then analyzed under varying noise level conditions. Second, experiments were run on real corrupted data (ISD) by training the models on the cleanest recordings and evaluating their performance on the noisiest recordings. Finally, the distribution of DSF filter weights predicted by a subset of the trained models was analyzed.

### Performance of Existing Methods Degrades Under Channel Corruption

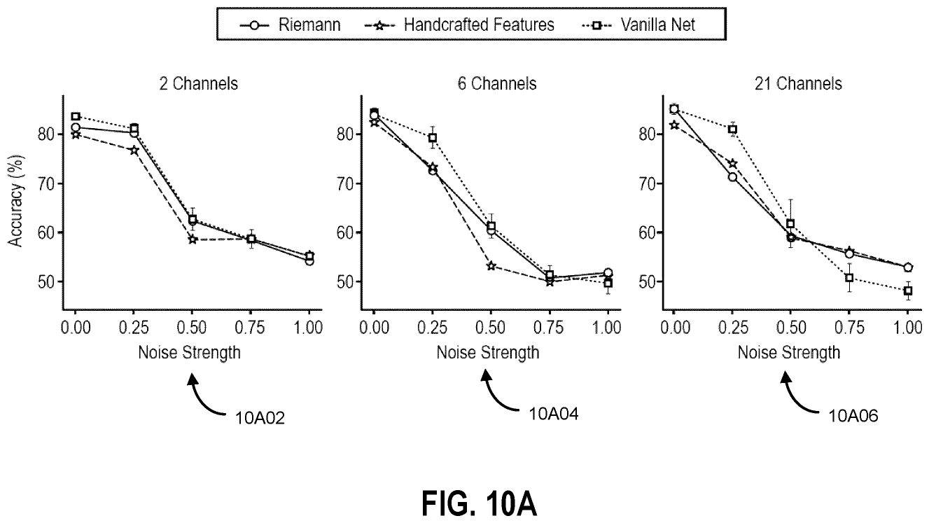

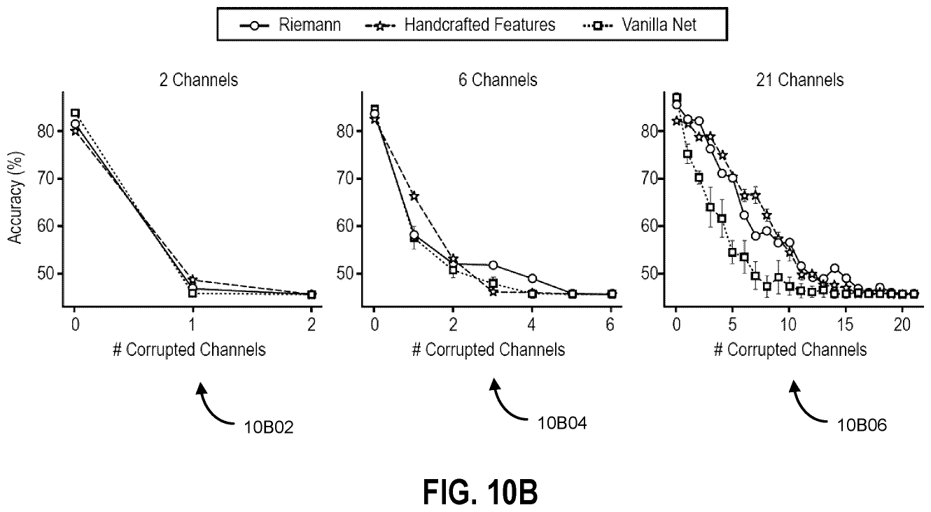

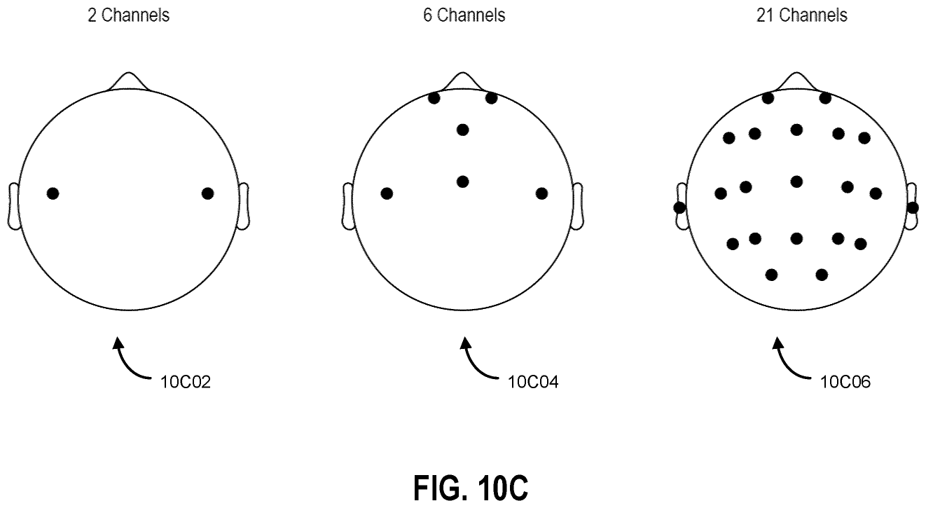

FIG. 10A illustrates the impact of noise strength on pathology detection performance of standard models where the η noise strength parameter was varied given a constant channel corruption probability of 50%, according to some embodiments. The models include simulations on 2 channels (10A02), 6 channels (10A04), and 21 channels (10A06).

FIG. 10B illustrates the impact of noise strength on pathology detection performance of standard models where the number of corrupted channels was varied given a constant noise strength of 1, according to some embodiments. The models include simulations on 2 channels (10B02), 6 channels (10B04), and 21 channels (10B06).

FIG. 10C illustrates the placement of 2 channels (10C02), 6 channels (10C04), and 21 channels (10C06), on the scalp of a user, according to come embodiments.

Impact of channel corruption on pathology detection performance of standard models. A filter-bank Riemannian geometry pipeline (circle), a random forest on handcrafted features (star) and a standard ShallowNet architecture (square) on the TUAB dataset, given montages of 2 (T3, T4), 6 (Fp1, Fp2, T3, T4, Fz, Cz) or 21 (all available) channels were trained. Performance was then evaluated on artificially corrupted test data under two scenarios: (A) the η noise strength parameter was varied given a constant channel corruption probability of 50%, and (B) the number of corrupted channels was varied given a constant noise strength of 1. Error bars show the standard deviation over 3 models for handcrafted features and 6 models for neural networks. While traditional feature-based models fared slightly better than a vanilla neural network in some cases (FIG. 10B, 10B06), adding noise predictably degraded the performance of all three models.

The performance of three baseline approaches (Riemannian geometry, handcrafted features, and a “vanilla” net, i.e., ShallowNet without attention) trained on a pathology detection task as channels were artificially corrupted, for three different montages was measured to determine how standard EEG classification methods fare against channel corruption and whether can noise be compensated for by adding more channels if channels have a high probability of being corrupted at test time.

All three baseline methods performed similarly and suffered considerable performance degradation as stronger noise was added (FIG. 10A) and as more channels were corrupted (FIG. 10B). First, under progressively noisier conditions, adding more channels did not generally improve performance. Strikingly, adding channels even hampered the ability of the models to handle noise. Indeed, the impact of noise was much less significant for 2-channel models than for 6- or 21-channel models. The vanilla net performed slightly better than the other methods in low noise conditions, however it was less robust to heavy noise when using 21 channels.

Second, when an increasing number of channels was corrupted (FIG. 10B), using denser montages did improve performance, although by a much smaller factor than what might be expected. For instance, losing one or two channels with the 21-channel models only yielded a minor decrease in performance, while models trained on sparser montages lost as much as 30% accuracy. However, even when as many as 15 channels were still available (i.e., six corrupted channels), models trained on 21 channels performed worse than 2- or 6-channel models without any channel corruption, despite having access to much more spatial information on average. Interestingly, when models were trained on 21 channels, other feature-based methods were more robust to corruption than a vanilla net up to a certain point, however this did not hold for sparser montages.

These results indicate that some standard approaches cannot handle significant channel corruption at a satisfactory level, even when denser montages are available. Therefore, better tools are necessary to train noise-robust models.

These are example results to illustrate aspects of some embodiments described herein.

### Attention and Data Augmentation Mitigates Performance Loss Under Channel Corruption

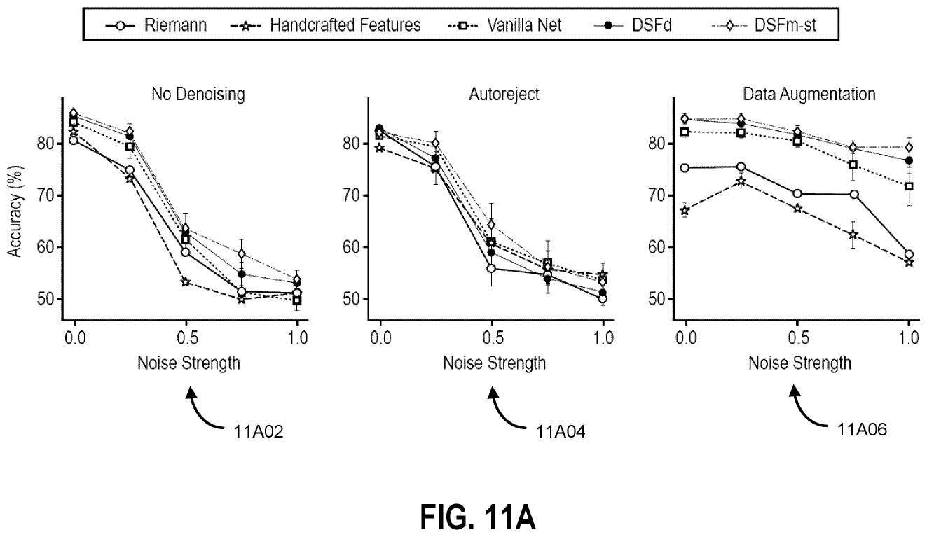

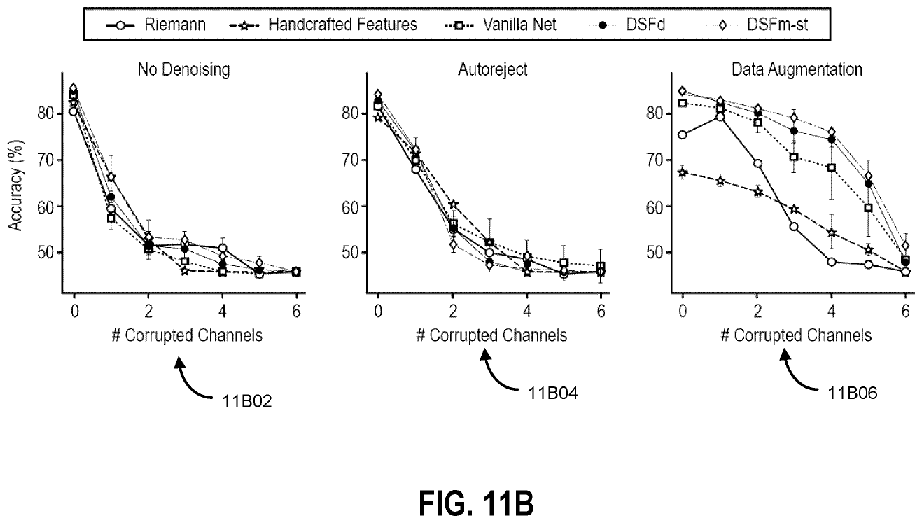

FIG. 11A illustrates the impact of noise strength on pathology detection performance for models coupled with no denoising strategy (11A02), Autoreject (11A04), and data augmentation (11A06) where the η noise strength parameter was varied given a constant channel corruption probability of 50%, according to some embodiments.

FIG. 11B illustrates the impact of noise strength on pathology detection performance for models coupled with no denoising strategy (11B02), Autoreject (11B04), and data augmentation (11B06) where the number of corrupted channels was varied given a constant noise strength of 1, according to some embodiments.

The per recording accuracy on the TUAB evaluation set (6-channel montage) was compared as (A) the η noise strength parameter was varied given a constant channel corruption probability of 50%, and (B) the number of corrupted channels was varied given a constant noise strength of 1. Error bars show the standard deviation over 3 models for handcrafted features and 6 models for neural networks. Using an automated noise handling method (Autoreject; 11A04 and 11B04) provided some improvement in noise robustness over using no denoising strategy at all (11A02 and 11B02). Data augmentation benefited all methods, but deep learning approaches and in particular DSF (11A06 and 11B06) yielded the best performance under channel corruption.

The performance of the models when combined with three denoising strategies for a fixed 6-channel montage was evaluated to determine what can be done to improve the robustness of standard EEG classification methods. This 6-channel montage (Fp1, Fp2, T3, T4, Fz, Cz) performed similarly to a 21-channel montage in no-corruption conditions (FIG. 10A and FIG. 10B) while being more representative of the sparse montages likely to be found in mobile EEG devices. Results on pathology detection (TUAB) are presented in FIG. 11A and FIG. 11B.

Without denoising, all methods tested showed a steep performance decrease as noise became stronger (FIG. 11A) or more channels were corrupted (FIG. 11B). Automated noise handling (11A04 and 11B04) reduced differences between methods when noise strength was increased (FIG. 11A), and helped marginally improve robustness when only one or two channels were corrupted (FIG. 11B). However, it is only with data augmentation that clear performance improvements could be obtained, allowing all methods to perform considerably better in the noisiest settings (11A06 and 11B06). Performance of traditional baselines was degraded however in low noise conditions. Neural networks, in contrast, saw their performance increase the most across noise strengths and numbers of corrupted channels. Whereas their performance decreased by at least 34.6% when going from no noise to strongest noise with the other strategies, training neural networks with data augmentation reduced performance loss to 5.3-10.5% on average. The DSF models improved performance further still over the vanilla ShallowNet by yielding an improvement of e.g., 1.8-7.5% across noise strengths. Finally, adding the matrix logarithm and the soft-thresholding nonlinearity (DSFm-st) yielded marginal improvements over DSFd. Under strong noise corruption (n=1) our best performing model (DSFm-st+data augmentation) yielded an accuracy improvement of 29.4% over the vanilla net without denoising. Overall, this suggests that learning end-to-end to both predict and handle channel corruption at the same time is key to successfully improving robustness.

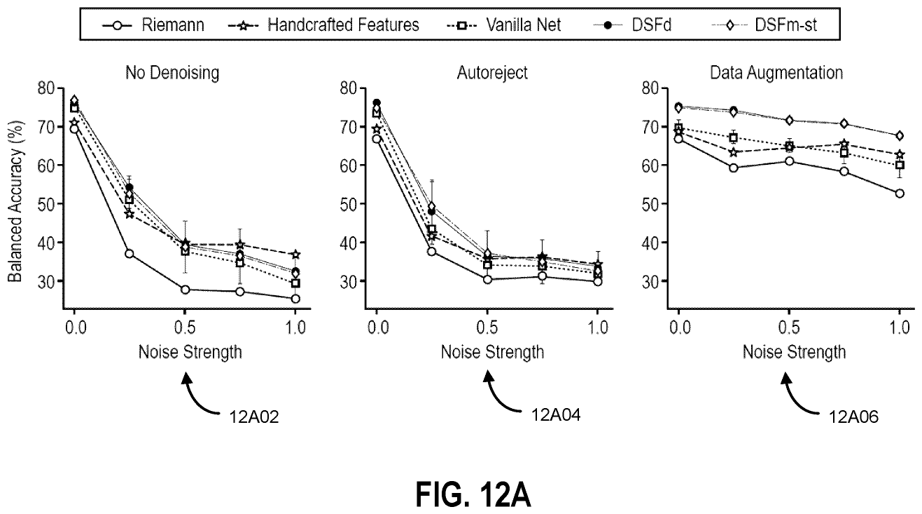

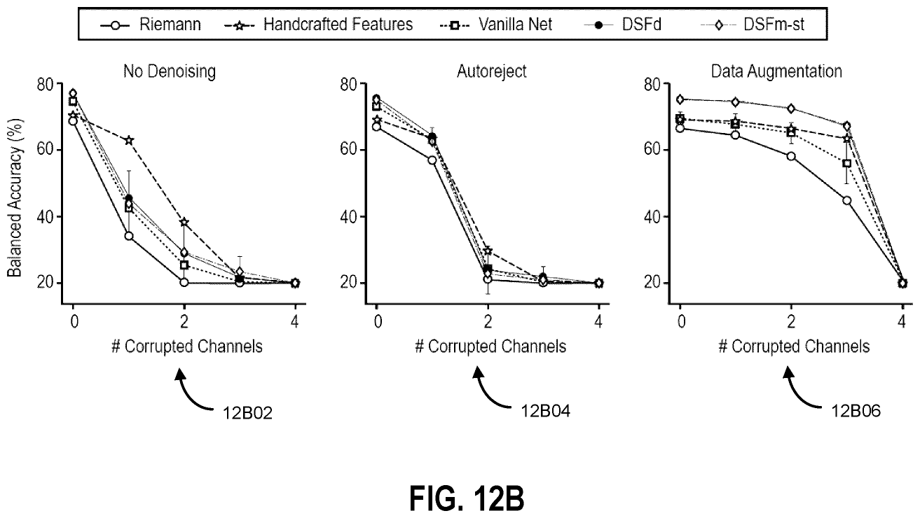

FIG. 12A illustrates the impact of noise strength on sleep staging performance for models coupled with no denoising strategy (12A02), Autoreject (12A04), and data augmentation (12A06) where the η noise strength parameter was varied given a constant channel corruption probability of 50%, according to some embodiments.

FIG. 12B illustrates the impact of noise strength on sleep staging performance for models coupled with no denoising strategy (12B02), Autoreject (12B04), and data augmentation (12B06) where the number of corrupted channels was varied given a constant noise strength of 1, according to some embodiments.

The test balanced accuracy on PC18 (4-channel montage) was compared as the η noise strength parameter was varied given a constant channel corruption probability of 50% (FIG. 12A) and the number of corrupted channels was varied given a constant noise strength of 1 (FIG. 12B). Error bars indicate the standard deviation over 3 models for handcrafted features and 4 models for neural networks. Similarly to FIG. 11A and FIG. 11B, automated noise handling provided a marginal improvement in noise robustness in some cases, data augmentation yielded a performance boost for all methods, while a combination of data augmentation and DSF consistently improved performance under channel corruption (12A02 and 12B02, lines which overlap) led to the best performance under channel corruption.

Next, this analysis was replicated on sleep staging task using the PC18 dataset (FIG. 12A and FIG. 12B). Similarly to TUAB results, not using a denoising strategy led to a steep decrease in performance. Interestingly, the Riemannian pipeline did not perform as well as other methods, while the handcrafted features baseline yielded higher robustness above a noise strength of 0.5. Once more, Autoreject leveled out differences between the different methods and boosted performance under single-channel corruption, but otherwise Autoreject overall did not improve performance clearly, and actually harmed the performance as compared to training models without denoising. Data augmentation, in contrast again, helped improve the robustness of all methods. Interestingly, it benefitted non-deep learning approaches more than in pathology detection, yielding for instance a similar performance for both handcrafted features and vanilla StagerNet. DSF remained the most robust though, with both the DSFd and DSFm-st variant consistently outperforming all other methods. The performance of these two methods was highly similar, producing mostly overlapping lines (FIG. 12A and FIG. 12B).

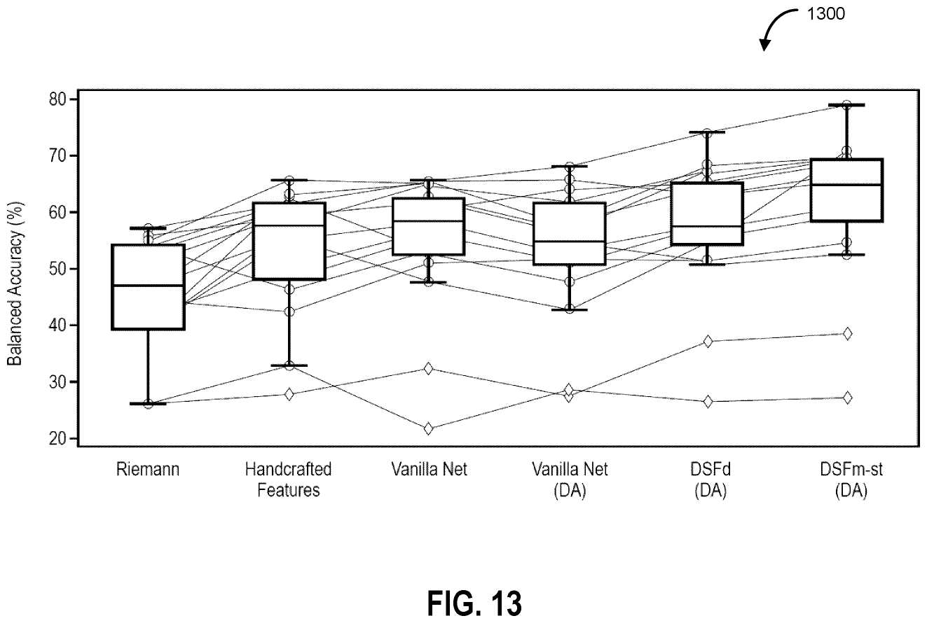

FIG. 13 illustrates recording-wise sleep staging results on ISD (1300), according to some embodiments. Test balanced accuracy is presented for the Riemann, handcrafted features and vanilla net models without a denoising strategy, and for the vanilla net, DSFd and DSFm-st models with data augmentation (DA), according to some embodiments. Each point represents the average performance obtained by models with different random initializations (1, 3 and 9 initializations for Riemann, hand-crafted features and deep learning models, respectively) on each recording from the test set of ISD. Lines represent individual recordings. The best performance was obtained by combining data augmentation with DSF with logm(cov) and soft-thresholding (DSFm-st).

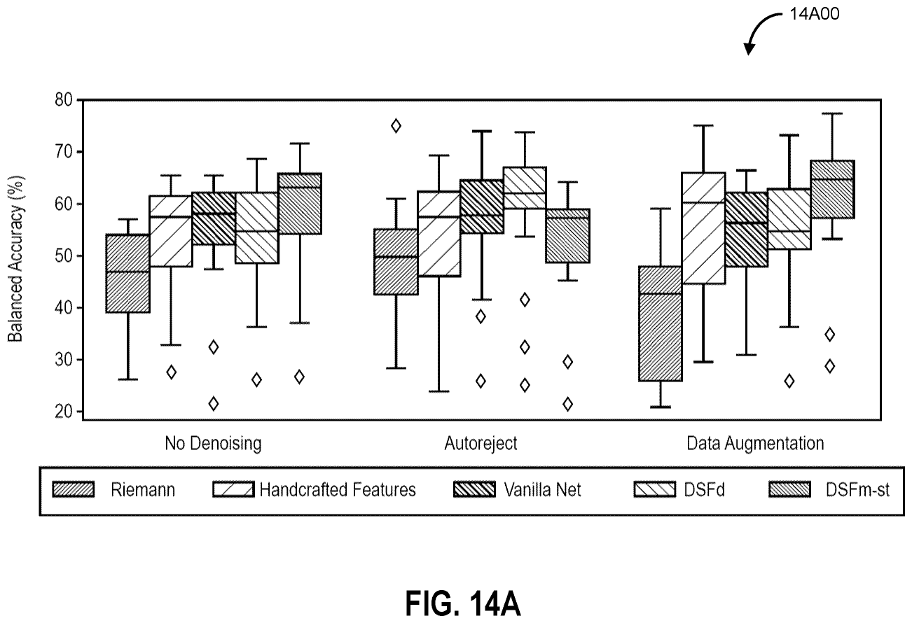

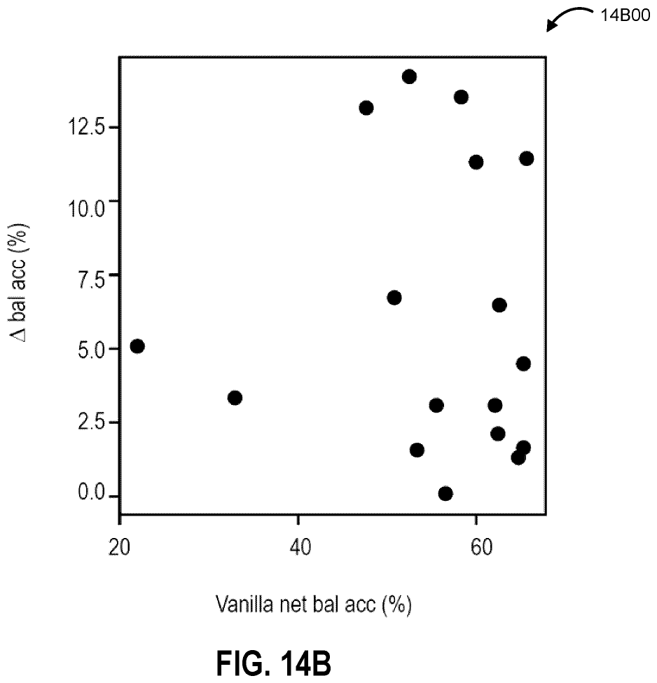

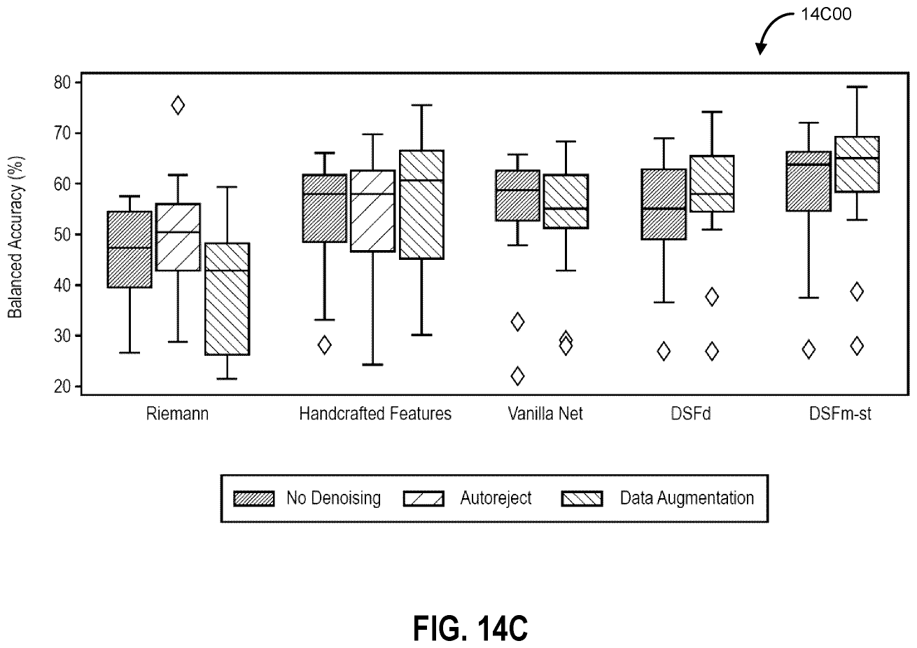

FIG. 14A illustrates the recording-wise sleep staging results on ISD showing test balanced accuracy for models coupled with (1) no denoising strategy, (2) Autoreject and (3) data augmentation (14A00), according to some embodiments. FIG. 14B illustrates the recording-wise sleep staging results on ISD showing that good performance is obtained by combining data augmentation with DSF with logm(cov) and soft-thresholding (DSFm-st) (14B00), according to some embodiments. Per-recording improvement in sleep staging performance obtained with DSF on ISD. The x-axis is the test balanced accuracy obtained by a vanilla net and the y-axis is the difference between the performance of DSF with logm(cov) and soft-thresholding and the performance of a vanilla net. Each point represents the average performance obtained by 9 models (random initializations) of a recording from the test set of ISD. All recordings saw an increase in performance.

FIG. 14C illustrates the performance of the baseline models combined with the different noise handling methodologies (14C00), according to some embodiments.

The same sleep staging models were trained as above on the cleanest recordings of ISD (4-channel mobile EEG), and their performance was evaluated on the 17 most corrupted recordings of the dataset to verify whether results hold under more intricate, naturally occurring corruption such as found in at-home settings. Results are presented in FIG. 13. As above, the Riemann approach did not perform well, while the handcrafted features approach was more competitive with vanilla StagerNet without denoising. Without denoising, handcrafted features, a vanilla StagerNet and DSFd performed similarly, however the use of the full covariance and soft-thresholding (DSFm-st) marginally improved the average performance. However, contrary to the above experiments, noise handling alone did not improve the performance of our models. Data augmentation was even detrimental to the Riemann and vanilla net models on average (see FIG. 14C). Combined with dynamic spatial filters (DSFd and DSFm-st) though, data augmentation helped improve performance over other methods. For instance, DSFm-st with data augmentation yielded a median balanced accuracy of 65.0%, as compared to 58.4% for a vanilla network without denoising. Performance improvements were as high as 14.2% when looking at individual sessions. Importantly, all recordings saw an increase in performance, showing the ability of our proposed approach to improve robustness in noisy settings.

Taken together, the experiments on simulated and natural channel corruption demonstrate that a strategy combining an attention mechanism and data augmentation yields higher robustness than traditional baselines and existing automated noise handling methods.

### Attention Weights are Interpretable and Correlate With Signal Quality

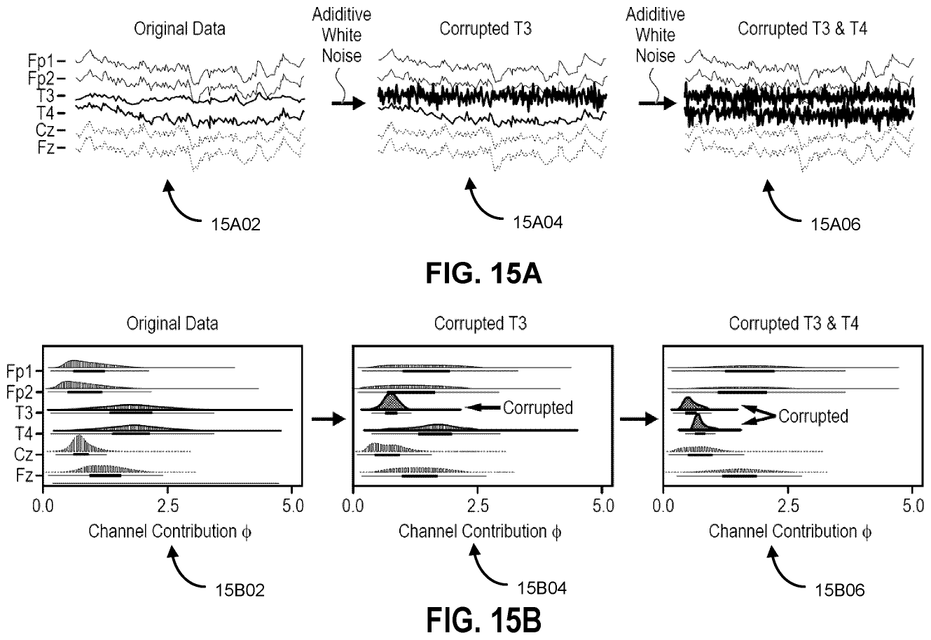

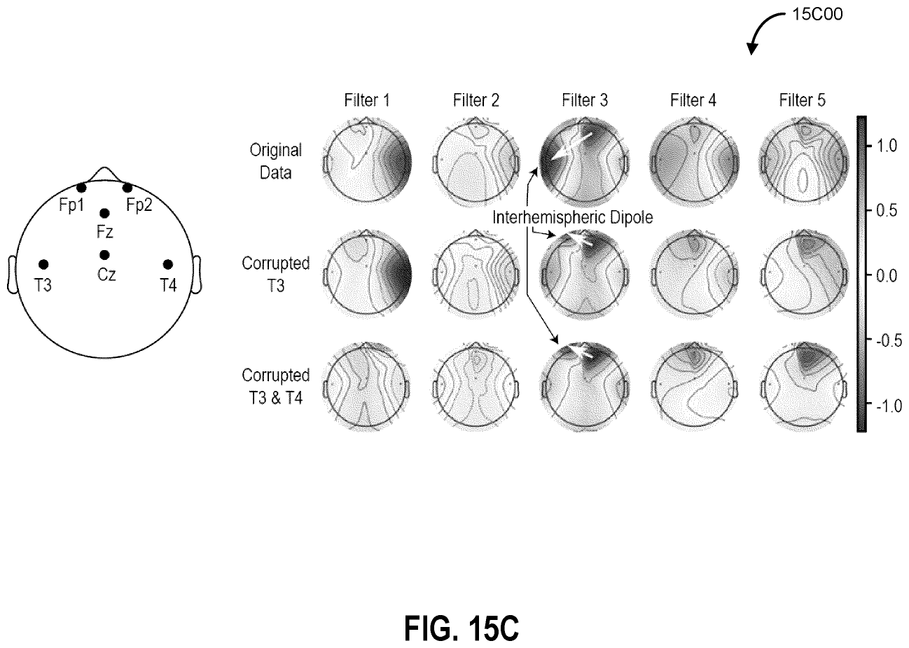

The foregoing demonstrate that DSF with data augmentation can lead to higher classification performance than “no denoising” and Autoreject baselines on both pathology detection and sleep staging tasks, under simulated and real-world channel corruption. The effective channel importance ϕᵢ of each EEG channel i to the spatial filters over the TUAB evaluation set was analyzed to test whether the behavior of the module can be explained by inspecting its internal functioning. If so, in addition to improving robustness, DSF may also be used to dynamically monitor the importance of each incoming EEG channel, providing an interesting “free” insight into signal quality. Results are shown in FIG. 15A, FIG. 15B, and FIG. 15C.

FIG. 15A illustrates the corruption process carried out to investigate the effective channel importance and spatial filters predicted by the DSF module trained on pathology detection, according to some embodiments. Three scenarios on the TUAB evaluation set were compared: no added corruption (15A02), only T3 is corrupted (15A04), and both T3 and T4 are corrupted (15A06). The process was carried out by replacing a channel with white noise (˜ [symbol] (20,50)), as illustrated with a single 6-s example window.

FIG. 15B illustrates the distribution of channel contribution values ϕ using density estimate and box plots obtained when investigating the effective channel importance and spatial filters predicted by the DSF module trained on pathology detection, according to some embodiments. Three scenarios on the TUAB evaluation set were compared: no added corruption (15B02), only T3 is corrupted (15B04), and both T3 and T4 are corrupted (15B06).

FIG. 15C illustrates a subset of the spatial filters (median across all windows) plotted as topomaps for the three scenarios (15C00), according to some embodiments. Corrupting T3 overall reduced the effective importance attributed to T3 and slightly boosted T4 values, while corrupting both T3 and T4 led to a reduction of ϕ for both channels, but to an increase for the other channels. This change was also reflected in the overall topography: dipole-like patterns (indicated by white arrows) were dynamically modified to focus on clean channels (e.g., Filter 3).

Overall, the more usable (i.e., noise free) a channel was, the higher its effective channel importance di was relative to those of other channels. For instance, without any additional corruption, the DSF module focused its attention on channels T3 and T4 (FIG. 15B, first column), known to be highly relevant for pathology detection. However, when channel T3 was replaced with white noise, the DSF module reduced its attention to T3 and instead further increased its attention on other channels (15B04). When both T3 and T4 were corrupted, on the other hand, the module reduced its attention on both channels, and leveraged the remaining channels instead, i.e., mostly Fp1 and Fp2 (15B06). Interestingly, this change is reflected by the topography of the predicted filters W_{DSF} (FIG. 15C): for instance some dipolar filters computing a difference between left and right hemispheres were dynamically adapted to rely on Fp1 or Fp2 instead of T3 or T4 (e.g., filters 1, 3, 5). The network has learned to ignore corrupted data and to focus its “attention” on the good EEG channels, and to do so in a way that preserves the “meaning” of each virtual channel.

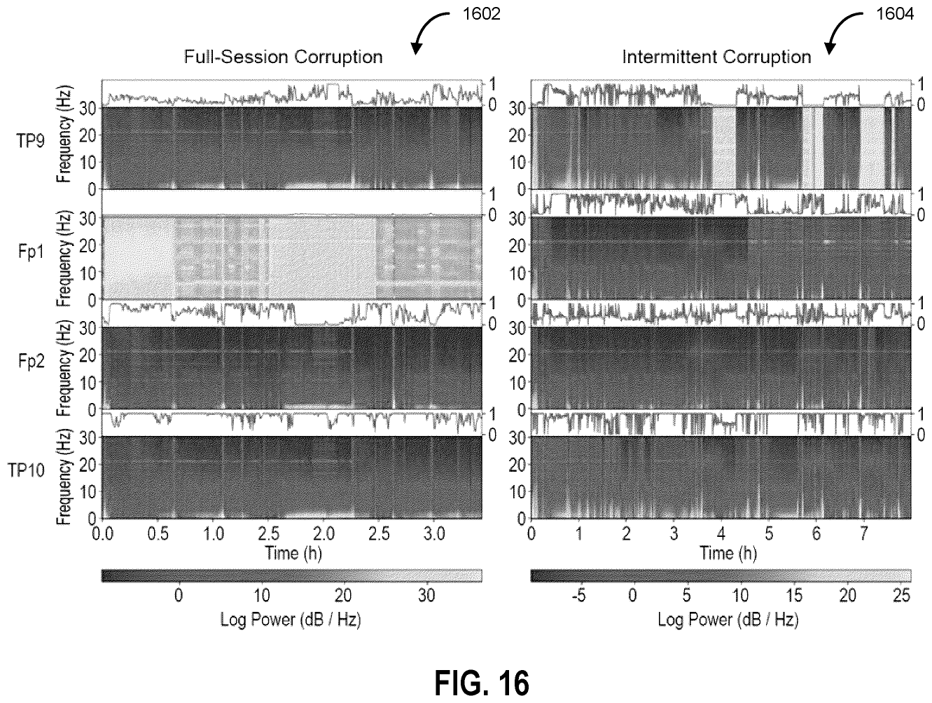

FIG. 16 illustrates normalized effective channel importance \hat{ϕ} predicted by the DSF module on two ISD sessions with naturally-occurring channel corruption (corruption throughout (1602), and intermittent corruption (1604)), according to some embodiments. Each column represents the log-spectrogram of the four EEG channels of one recording (Welch's periodogram on 30-s windows, using 2-s windows with 50% overlap). The line above each spectrogram is the normalized effective channel importance \hat{ϕ}ᵢ (see Eq. 13), between 0 and 1, computed using a

DSFm-st model trained on ISD. When a channel is corrupted throughout the recording (1602, second row, as indicated by broad spectrum high power noise), DSF can mostly “ignore” it by predicting small weights for that channel. This results in \hat{ϕ}ᵢ values close to 0 for Fp1. When the corruption is intermittent (1604, first row), DSF can dynamically adapt its spatial filters to only ignore important channels when they are corrupted. This is the case for channel TP9 around hours 4, 6, and 7, where \hat{ϕ}ᵢ is again close to 0.

To further verify the interpretability of DSF's attention weights on naturally-corrupted real-world EEG data, the normalized effective channel importance metric was visualized alongside a time-frequency representation of the raw EEG in FIG. 16. The metric dropped to values close to zero when a channel suffered heavy corruption, e.g., Fp1 throughout the recording (left column) and TP9 intermittently (right column). These results again illustrate the capacity of DSF to ignore corrupted data, but also highlight its capacity to dynamically adapt to changing noise characteristics.

### Deconstructing the DSF Module

By comparing DSF to simpler interpolation-based methods, the capacity of the DSF module to improve robustness to channel corruption and provide interpretable attention weights, is observed. DSF can be understood as a more complex version of a simple attention-based model that decides how much each input EEG channel should be replaced by its interpolated version.

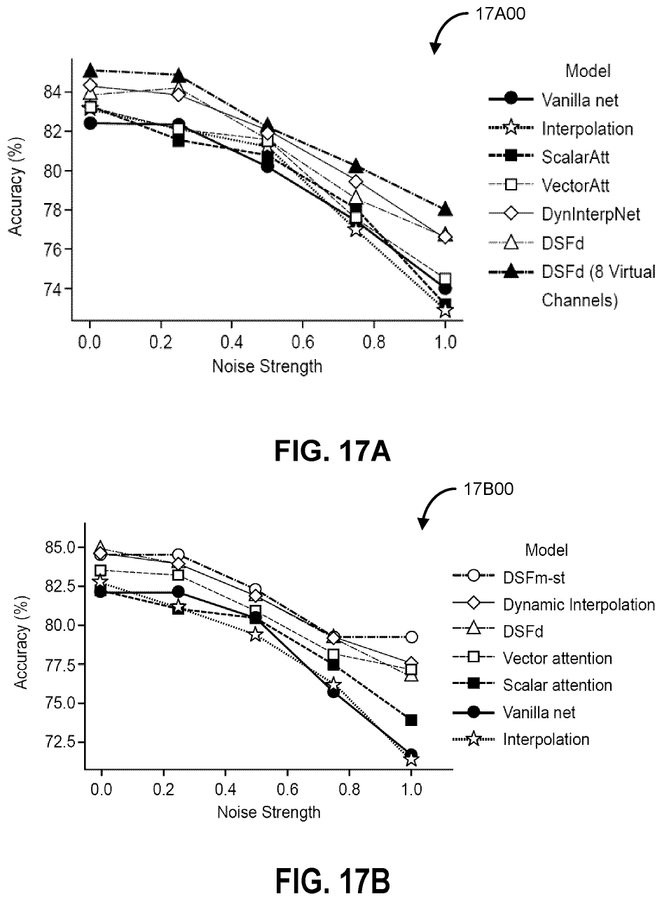

With this connection in mind, an ablation study was performed to understand the importance of each additional mechanism leading to the formulation of the DSF module. FIG. 17A and FIG. 17B each illustrate a set of the performance of the different attention module variations trained on the pathology detection task with data augmentation, under different noise strengths (17A00 and 17B00), according to come embodiments.

FIG. 17A and FIG. 17B each illustrate a set of the performance of different attention module architectures on the TUAB evaluation set under increasing channel corruption noise strength, according to come embodiments. Each line represents the average of 6 models (2 random seeds, 3 random splits). Models that dynamically generate spatial filters, such as DSF, outperform simpler architectures across noise levels.

Naive interpolation of each channel based on the C−1 others (white star) performed similarly to or worse than the vanilla ShallowNet model (black circle) across noise strengths. Introducing a single attention weight (black square) to control how much channels should be mixed with their interpolated version only improved performance for noise strengths above 0.5. Using one attention weight per channel (white square) further improved performance, this time across all noise strengths. The addition of dynamic interpolation (white diamond), in which both the attention weights and an interpolation matrix are generated based on the input EEG window, yielded an additional substantial performance boost. Relaxing the constraints on the interpolation matrix and adding a bias vector to obtain DSFd (white triangle) led to similar performance. Finally, the performance across noise strengths can be further improved by adding additional virtual channels (e.g., C′=8, black triangle). The addition of the soft-thresholding non-linearity and the use of the matrix logarithm of the covariance (DSFm-st, white circle) further yielded performance improvements.

Together, these results show that combining channel-specific interpolation and dynamic prediction of interpolation matrices are useful to outperform simpler attention module formulations. Generalizing the architecture so that it performs spatial filtering instead of spatial interpolation (i.e., from dynamic interpolation to the affine transformation of DSF) further improves performance while yielding a simple formulation (Eq. 2) and allowing the use of more virtual channels. Performance can be further improved by providing the full covariance matrix as input to the attention module and encouraging the model to produce 0-weights with a non-linearity.

### General Discussion

Dynamic Spatial Filtering (DSF), a new method to handle channel corruption in EEG based on an attention mechanism architecture and a data augmentation transform, was introduced. Plugged into a neural network whose input has a spatial dimension (e.g., EEG channels), DSF can predict spatial filters that allow the model to dynamically focus on important channels and ignore corrupted ones. DSF shares links with interpolation-based methods traditionally used in EEG processing to recover bad channels, but in contrast to these approaches does not require separate preprocessing steps that are often expensive with dense montages or poorly adapted to sparse one. Moreover, DSF can outperform feature-based approaches and automated denoising pipelines under simulated corruption on two large public datasets and in two different predictive tasks. Similar results can be obtained on a smaller dataset of mobile sparse EEG with strong natural corruption, demonstrating the applicability of the approach to challenging at-home recording conditions. Finally, the inner functioning of DSF can be inspected using a simple measure of effective channel importance and topographical maps. Overall, DSF can be computationally lightweight and easy to implement, and can improve robustness to channel corruption in sparse EEG settings.

### Handling EEG Channel Loss With Existing Denoising Strategies

The systems and methods described herein, in some embodiments, specifically focus on the problem of channel corruption in sparse montages.

Attention and data augmentation mitigates performance loss under channel corruption demonstrates that adding more EEG channels does not necessarily make a classifier more robust to channel loss. In fact, the opposite was observed: a model trained on two channels can outperform 6- and 21-channel models under heavy channel corruption (FIG. 10A). This can be explained by two phenomena. First, increasing the number of channels increases (linearly or superlinearly (in the case of handcrafted features that look at channel pairs, e.g., phase locking value)) the input dimensionality of classifiers, making them more likely to overfit the training data. Tuning regularization hyperparameters can help with this, but does not solve the problem by itself. Second, in vanilla neural networks, the weights of the first spatial convolution layer, i.e., the spatial filters applied to the input EEG, are fixed. If one of the spatial filter outputs relies mostly on one specific (theoretically) important input channel, e.g., T3, and that this input channel is corrupted, all successive operations on the resulting virtual channel may carry noise as well. When models are trained with more input channels, the learned spatial filters are likely to focus on specific channels that happen to be the most informative. When the critical channels are missing at inference time the model can suffer a strong performance loss. This highlights the importance of dynamic re-weighting: with DSF, alternative spatial filters can be found when a theoretically important channel is corrupted, and even completely ignore a corrupted channel if it contains no useful information.

Since adding channels is not on its own a solution, can traditional EEG denoising techniques help handle the channel corruption problem? A fixed threshold on a relevant descriptor of signal quality (e.g., amplitude, variance or spectral slope) used to identify bad channels window-by-window requires making non-trivial choices such as: Which descriptor to use? How should the threshold values be selected? How are bad channels handled once they have been identified? Moreover, this approach is likely to perform suboptimally as different EEG hardware, channel and reference positions, preprocessing steps and recording conditions, especially in out-of-the-lab settings, all have an impact on the power and morphology of the signals. As a result, fixed threshold values will fail to catch actual noise (or be too strict) in others. Instead, adapting thresholds in a data-driven manner is a more sophisticated approach. This is the basis for Autoreject which selects amplitude thresholds using a cross-validation procedure and interpolates bad channels using head geometry. In the foregoing, interpolation-based denoising did help handle the channel corruption problem but only marginally (center column of FIG. 11A, FIG. 11B, FIG. 12A, and FIG. 12B). The relative ineffectiveness of this approach can be explained by the very low number of available channels (4 or 6) which likely harmed the quality of the interpolation. This exposes the limitations of interpolation-based methods when working with few channels. Still, there are other reasons why interpolation-based methods might not be optimal in settings like the ones studied in this paper. For instance, completely replacing a noisy channel by its interpolated version means that (1) any remaining usable information in this channel is discarded and (2) noise contained in the other (non-discarded) channels will end up in the interpolated channel. On top of this, automated denoising techniques such as Autoreject require an additional preprocessing step at both training and inference time. In machine learning pipelines where only the downstream task performance matters but not the morphology of the signals (e.g., as opposed to evoked potential studies), these techniques are therefore preferably avoided. This criticism can apply to reconstruction-based methods as well, for which deep learning-based interpolation with e.g., a GAN can be costly. An end-to-end solution such as DSF takes care of both these issues by (1) dynamically deciding how much of each channel should be used and (2) not requiring any extra steps at training or inference time.

As long as developing an invariance to the type of noise in question is useful to the downstream task (e.g., for handling other types of noise (e.g., artifacts) and/or in denser montage settings), DSF may help. Any pattern that can be detected using the DSF input representation (e.g., the vectorized covariance matrix) can theoretically be accounted for in the choice of spatial filters. Different choices of input representations could further be leveraged based on the type of noise expected. As for denser montages, nothing precludes DSF to also work with higher numbers of EEG channels. Its capacity to leverage spatial information might actually improve. However, to avoid an explosion in the number of parameters of its internal MLP, careful hyperparameter search might be necessary and structured prediction strategies (e.g., enforcing structure between the predicted spatial filters) might be useful.

Finally, an interesting case to consider is when tasks can be performed accurately with a single good channel, e.g., sleep staging. While a single-channel model perform as well as a multi-channel model, without the need to worry about the challenges discussed above may be true if we have access to a reliably good channel, as soon as it is corrupted (e.g., in real-world mobile EEG settings) it can no longer be used by the model. An ensemble of single-channel models requires knowing both which channel to focus on and when, which is not trivial and requires additional logic and processing pipeline components. Moreover, to improve upon such a model by making use of spatial information the model should be trained on all possible combinations of good channels, which can quickly become prohibitive. DSF offers a compelling solution to the challenges encountered with single-channel models thanks to its end-to-end dynamic reweighting capabilities.

### Impact of the Input Spatial Representation

The representation used by the DSF module constrains the types of patterns that can be leveraged to produce spatial filters. For instance, using the log-variance of each channel allows detecting large-amplitude corruption or artifacts, however can make the DSF model blind to subtler kinds of interactions between channels. These interactions can be very informative in certain cases, e.g., when one channel is corrupted by a noise source which also affects other channels but to a lesser degree.

The above suggest that models based on log-variance (DSFd) or vectorized covariance matrices (DSFm-st) were roughly equivalent in simulated conditions (FIG. 11A, FIG. 11B, FIG. 12A and FIG. 12B). This may be because the additive white noise used was not spatially correlated and therefore no spatial interactions could be leveraged by the DSF modules to identify noise. Additionally, the soft-thresholding non-linearity made it easier for the model to wipe out noisy channels, while the convex combination of signal and noise meant corrupted channels often still contained useful information. On naturally corrupted data however, using the full spatial information along with soft-thresholding was helpful to outperforming other methods (FIG. 13). This is likely because the noise in at-home recordings often correlated spatially and because corrupted channels, often containing mostly noise could completely ignored by DSF.

Related attention block architectures have used average-pooling or a combination of average- and max-pooling to summarize channels. Intuitively, average pooling should not result in a useful representation of input EEG channels, as EEG channels are often assumed to have zero-mean, or are explicitly highpass filtered to remove their DC offset. Max-pooling, on the other hand, can capture amplitude information that overlaps with second-order statistics, however it may not allow to differentiate between large transient artifacts that only affect a small portion of the window and more temporally consistent corruption. Experiments on TUAB (not shown) confirmed this in practice, with a combination of min- and max-pooling being less robust to noise than covariance-based models. From this perspective, vectorized covariance matrices are an ideal choice of spatial representation for dynamic spatial filtering of EEG. Other representations could be investigated such as correlation matrices. Ultimately, this representation itself could be learned, although this comes at the price of larger models.

### Impact of the Data Augmentation Transform

Data augmentation was critical to developing invariance to corruption (Attention and data augmentation mitigates performance loss under channel corruption). For instance, on simulated corruption, a vanilla neural network trained with the data augmentation transform gained considerable robustness once, even without an attention mechanism. As shown on naturally corrupted data (FIG. 13), data augmentation without attention negatively impacted performance. This phenomenon can likely be explained by the alignment between the data augmentation transform used at training time and the type of corruption expected at test time. In the simulations, spatially uncorrelated white noise was added to random channels both to synthesize corruption and to augment the data (with the difference that noise parameters were set recording-wise for corruption and window-wise for data augmentation). This likely helped neural networks perform well under this type of channel corruption. Moreover, traditional pipelines generally did not benefit from data augmentation as much as neural networks did, and even saw their performance degrade considerably in certain cases, e.g., in low noise conditions in pathology detection experiments and on the real-world data for the Riemann models. On naturally corrupted data however, not only did the models see a different type of corruption during training, but the variability of noise at testing time was much greater than in simulations. Therefore, even with data augmentation, a vanilla net did not generalize well to natural corruption, whereas the ability of the DSF module to handle channel corruption can be applied to other types of noise too.

Nonetheless, this highlights the role of data augmentation transforms in developing robust representations of EEG. Well characterized data augmentation transforms may be important to learning representations. Importantly though, the motivation behind the use of data augmentation is to evaluate methods under controlled corruption of experimental data not to reduce overfitting due to limited sample sizes like commonly done in deep learning. Ultimately, additive white noise transform could be combined with channel masking and shuffling and other potential corruption processes. Interestingly, it was found that performing data augmentation on the validation set too further improves robustness (results not shown).

### Interpreting Dynamic Spatial Filters to Measure Effective Channel Importance

The results in FIG. 15A, FIG. 15B, and FIG. 15C demonstrate that visualizing the spatial filters produced by the DSF module can reveal the spatial patterns a model learns to focus on (Attention weights are interpretable and correlate with signal quality). A measure of the importance of each input channel may also be easily obtained by computing the norm of each column of the spatial filter matrix. For instance, this “effective channel contribution” metric ¢ was reactive to channel corruption (FIG. 15A, FIG. 15B, and FIG. 15C). To further facilitate the interpretation of channel contribution, e.g., in real-time settings where the user might want to monitor signal quality, a relative channel contribution metric ϕᵣₑₗ∈[0, 1] may be obtained by dividing each di by the maximum across channels.

A higher ϕ may indicate higher effective importance of a channel for the downstream task. For instance, temporal channels were given a higher importance in the pathology detection task. Similarly, in real-world data, low ϕ values were given to a channel whenever it was corrupted (FIG. 16).

Importantly, ϕ is not a strict measure of signal quality but more of channel usefulness: there could be different reasons behind the boosting or attenuation of a channel by the DSF module. Naturally, if a channel is particularly noisy, its contribution might be brought down to zero to avoid contaminating virtual channels with noise. Conversely though, if the noise source behind a corrupted channel is also found (but to a lesser degree) in other channels, the corrupted channel could also be used to regress out noise and recover clean signals. In other words, ϕ can reflect the importance of a channel conditionally to others.

Finally, using DSF to obtain a measure of channel usefulness may open the door to DSF being used in non-machine learning settings. For instance, once a neural network is trained with DSF, its effective channel importance values can be reused as an indicator of signal quality on similar data (e.g., data collected with the same or similar hardware). Such a signal quality metric can be helpful during data collection, or to know which parts of the recording should be kept for analysis.

### Practical Considerations

When faced with channel corruption in a predictive task, the preferred modelling and denoising strategies will depend on the number of available channels, as well as on assumptions about the stationarity of the noise. When using sparse montages, different solutions can lead to good results. When less can be assumed about the predictive task, e.g., corruption might be non-stationary o r spatial information is likely important, DSF with data augmentation is an effective way to make a neural network noise-robust. DSF with data augmentation might also be a promising end-to-end solution (in this case, the number of parameters of the module can be controlled by e.g., selecting log-variance as the input representation or reducing dimensionality by using fewer spatial filters than there are input channels) in cases where introducing a separate preprocessing step is not desirable.

### Further Applications of DSF

DSF can be used to train deep neural networks for speech recognition models, for example, using an array of microphones.

Sleep data above focused on window-wise decoding, i.e., it does not aggregate larger temporal context, but directly mapped each window to a prediction. However, modeling these longer-scale temporal dependencies can help sleep staging performance significantly. Despite a slight performance decrease, window-wise decoding offers a realistic setting to test robustness to channel corruption, while limiting the number of hyperparameters and the computational cost of the experiments. In practice, the effect of data corruption by far exceeded the drop in performance caused by using slightly simpler architectures.

The data augmentation and the noise corruption strategies exploited herein employ additive Gaussian white noise. While this approach helped develop noise robust models, spatially non-correlated additive white noise represents an “adversarial scenario”. Indeed, under strong white noise the information in higher frequencies is more likely to be lost than with e.g., pink or brown noise. In addition, the absence of spatial noise means spatial filtering can less easily leverage multi-channel signals to regress out noise. Some embodiments of DSF may work under different more varied and realistic types of channel corruption conditions. Additive white noise as a data augmentation can help improve noise robustness.

DSF may be useful on tasks where fine-grained spatial patterns might be critical to successful prediction, e.g., brain age estimation. Other common EEG-based prediction tasks such as seizure detection might benefit from DSF.

### CONCLUSION

Dynamic Spatial Filtering (DSF), an attention mechanism architecture that improves robustness to channel corruption in EEG prediction tasks, was presented. Combined with a data augmentation transform, DSF can outperform automated noise handling procedures under simulated and real channel corruption on three datasets. Moreover, DSF can enable efficient end-to-end handling of channel corruption, work with few channels, be interpretable and does not require expensive preprocessing. DSF can be applied to other types of data with spatial information and high probability of channel corruption (e.g., multivariate speech recordings). DSF can be used on filter-bank representations (e.g., the output of a temporal convolution layer) to handle even more precisely noise sources with different spectral signatures. This method may improve the reliability of EEG processing in challenging non-traditional settings such as user-administered, at-home recordings.

### Closing Remarks

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the foregoing discussion, numerous references were made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The following discussion provides many example embodiments. Although each embodiment represents a single combination of inventive elements, other examples may include all possible combinations of the disclosed elements. Thus if one embodiment comprises elements A, B, and C, and a second embodiment comprises elements B and D, other remaining combinations of A, B, C, or D, may also be used.

The term “connected” or “coupled to” may include both direct coupling (in which two elements that are coupled to each other contact each other) and indirect coupling (in which at least one additional element is located between the two elements).

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements. The embodiments described herein are directed to electronic machines and methods implemented by electronic machines adapted for processing and transforming electromagnetic signals which represent various types of information. The embodiments described herein pervasively and integrally relate to machines, and their uses; and the embodiments described herein have no meaning or practical applicability outside their use with computer hardware, machines, and various hardware components. Substituting the physical hardware particularly configured to implement various acts for non-physical hardware, using mental steps for example, may substantially affect the way the embodiments work. Such computer hardware limitations are clearly essential elements of the embodiments described herein, and they cannot be omitted or substituted for mental means without having a material effect on the operation and structure of the embodiments described herein. The computer hardware is essential to implement the various embodiments described herein and is not merely used to perform steps expeditiously and in an efficient manner.

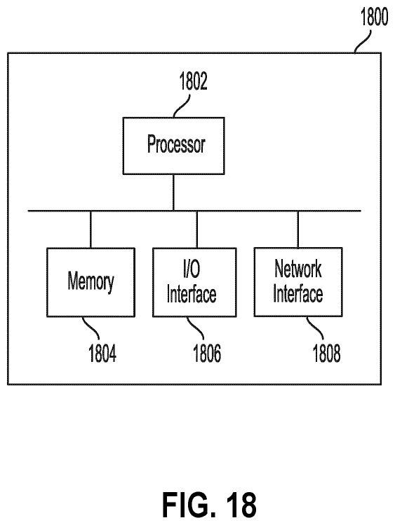

FIG. 18 is a schematic diagram of computing device 1800, exemplary of an embodiment. As depicted, computing device 1800 includes at least one processor 1802, memory 1804, at least one I/O interface 1806, and at least one network interface 1808. Computing device 1800 can be used to implement operations of processes and systems described herein.

Each processor 1802 may be, for example, any type of general-purpose microprocessor or microcontroller, a digital signal processing (DSP) processor, an integrated circuit, a field programmable gate array (FPGA), a reconfigurable processor, a programmable read-only memory (PROM), or any combination thereof.

Memory 1804 may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like.

Each I/O interface 1806 enables computing device 1800 to interconnect with one or more input devices, such as a keyboard, mouse, camera, touch screen and a microphone, or with one or more output devices such as a display screen and a speaker.

Each network interface 1808 enables computing device 1800 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these.

Computing device 1800 is operable to register and authenticate users (using a login, unique identifier, and password for example) prior to providing access to applications, a local network, network resources, other networks and network security devices. Computing devices 1800 may serve one user or multiple users.

For simplicity only one computing device 1800 is shown but system may include more computing devices 1800 operable by users to access remote network resources and exchange data. The computing devices 1800 may be the same or different types of devices. The computing device 1800 may include at least one processor, a data storage device (including volatile memory or non-volatile memory or other data storage elements or a combination thereof), and at least one communication interface. The computing device components may be connected in various ways including directly coupled, indirectly coupled via a network, and distributed over a wide geographic area and connected via a network (which may be referred to as “cloud computing”).

For example, and without limitation, the computing device may be a server, network appliance, set-top box, embedded device, computer expansion module, personal computer, laptop, personal data assistant, cellular telephone, smartphone device, UMPC tablets, video display terminal, gaming console, electronic reading device, and wireless hypermedia device or any other computing device capable of being configured to carry out the methods described herein.

Although the embodiments have been described in detail, it should be understood that various changes, substitutions and alterations can be made herein without departing from the scope as defined by the appended claims.

Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

As can be understood, the examples described above and illustrated are intended to be exemplary only. The scope is indicated by the appended claims.

1. A method of using a neural network to dynamically reweigh a plurality of channels according to relevance given a learning task or channel corruption, the method comprising:
receiving a dataset from a plurality of channels, each channel of the plurality of channels comprising data;
extracting a representation of the dataset or the plurality of channels;
predicting a dynamic spatial filter from the representation of the dataset or the plurality channels using a neural network;
applying the dynamic spatial filter to dynamically reweigh each of the channels of the plurality channels; and
performing a learning task using the reweighed channels and a second neural network.

2. (canceled)

3. (canceled)

4. (canceled)

5. (canceled)

6. The method of claim 1 wherein:
the applying the dynamic spatial filter comprises applying the dynamic spatial filter to at least one of input of a first layer of the second neural network and output of a layer of the second neural network.

7. (canceled)

8. (canceled)

9. The method of claim 1 wherein:
the plurality of channels comprises a plurality of sensors or a plurality of subdivisions of one sensor;
wherein the method comprises:
performing measurements for the dataset using the plurality of sensors or the one sensor.

10. (canceled)

11. The method of claim 1 wherein:
the dataset comprises output of a layer of the second neural network; and
the performing a learning task using the reweighed channels and the second neural network comprises providing the reweighed channels to at least one subsequent layer to the layer of the second neural network.

12. (canceled)

13. (canceled)

14. The method of claim 1 wherein:
the dynamic spatial filter comprises at least one of a weight matrix and a bias vector.

15. (canceled)

16. The method of claim 1 further comprising:
visualizing the dynamic spatial filter at an interface.

17. (canceled)

18. (canceled)

19. The method of claim 1 further comprising:
identifying an optimal location for hardware corresponding to at least one channel of the plurality of channels based in part on the dynamic spatial filter.

20. (canceled)

21. (canceled)

22 (canceled)

23. The method of claim 1 further comprising:
identifying a source space using the dynamic spatial filter.

24. The method of claim 1 further comprising:
using results of the learning task to adjust at least one trainable parameter of at least one of the neural network and the second neural network.

25. (canceled)

26. The method of claim 1 further comprising:
selectively transmitting at least one channel of the plurality of channels based in part on the dynamic spatial filter.

27. The method of claim 1 wherein:
the applying the dynamic spatial filter comprises transmitting a representation of the dynamically reweighed channels to perform the learning task, wherein the representation of the dynamically reweighed channels has reduced dimensions relative to the dynamically reweighed channels.

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. The method of claim 24 further comprising:
adding noise or channel corruption to the dataset or the plurality of channels prior to extracting a representation of the dataset or the plurality of channels.

61. (canceled)

62. A system for dynamically reweighing a plurality of channels according to relevance given a learning task or channel corruption using a neural network, the system comprising:
a plurality of channels, each channel of the plurality of channels comprising data; and
a computing device configured to:
receive a dataset from the plurality of channels;
extract a representation of the dataset or the plurality of channels;
predict a dynamic spatial filter from the representation of the dataset or the plurality of channels using a neural network;
apply the dynamic spatial filter to dynamically reweigh each of the channels of the plurality channels; and
perform a learning task using the reweighed channels and a second neural network.

63. (canceled)

64. (canceled)

65. (canceled)

66. (canceled)

67. The system of claim 62 wherein:
the apply the dynamic spatial filter comprises applying the dynamic spatial filter to at least one of input of a first layer of the second neural network and output of a layer of the second neural network.

68. (canceled)

69. (canceled)

70. The system of claim 62 wherein:
the plurality of channels comprises a plurality of sensors or subdivisions of one sensor; and
wherein the computing device is further configured to:
perform measurements for the dataset using the plurality of sensors or the one sensor channels.

71. (canceled)

72. The system of claim 62 wherein:
the dataset comprises output of a layer of the second neural network; and
the perform a learning task using the reweighed channels and the second neural network comprises providing the reweighed channels to at least one subsequent layer to the layer of the second neural network.

73. (canceled)

74. (canceled)

75. The system of claim 62 wherein:
the dynamic spatial filter comprises at least one of a weight matrix and a bias vector.

76. (canceled)

77. The system of claim 62 further comprising:
a display; and wherein
the computing device is further configured to:
visualize the dynamic spatial filter in real-time-on the display.

78. (canceled)

79. (canceled)

80. The system of claim 62 wherein:
the computing device is further configured to:
identify an optimal location for hardware corresponding to at least one channel of the plurality of channels based in part on the dynamic spatial filter.

81. (canceled)

82. (canceled)

83. (canceled)

84. The system of claim 62 wherein:
the computing device is further configured to:
identify a source space using the dynamic spatial filter.

85. The system of claim 62 wherein:
the computing device is further configured to:
adjust at least one trainable parameter of at least one of the neural network and the second neural network.

86. (canceled)

87. The system of claim 62 wherein:
the computing device is further configured to:
selectively transmit at least one channel of the plurality of channels based in part on the dynamic spatial filter.

88. The system of claim 62 wherein:
the apply the dynamic spatial filter comprises transmitting a representation of the dynamically reweighed channels to perform the learning task, wherein the representation of the dynamically reweighed channels has reduced dimensions relative to the dynamically reweighed channels.

89. (canceled)

90. (canceled)

91. (canceled)

92. The system of claim 85, wherein the computing device is further configured to:
add noise or channel corruption to the dataset or the plurality of channels prior to extracting a representation of the dataset or the plurality of channels.
